(19) United States
(12) Patent Application Publication
(10) Pub. No.: US 2025/0151686 A1
(43) Pub. Date: May 15, 2025

(54) METHODS OF INDUCING UNREDUCED APOSPOROUS OR DIPLOSPOROUS EMBRYO SAC FORMATION IN A SEXUAL ANGIOSPERM

(71) Applicant: UTAH STATE UNIVERSITY, Logan, UT (US)

(72) Inventor: John G. CARMAN, Smithfield, UT (US)

(21) Appl. No.: 18/838,582

(22) PCT Filed: Feb. 14, 2023
(86) PCT No.: PCT/US2023/062565

§ 371 Date: Aug. 14, 2024

Related U.S. Application Data
(60) Provisional application No. 63/310,092, filed on Feb. 14, 2022.

(51) Int. Cl. A01H 1/02; A01H 4/00

(52) U.S. Cl. CPC A01H 1/022; A01H 4/005

(57) ABSTRACT

The present invention discloses wild type genes that upon their down regulation unequivocally induce AES formation in sexual plants. Photomicrographs of apospory in the down regulated germplasm meet all four criteria. The identified genes and methods may be used as part of a genetic engineering strategy to convert sexual crops to apomixis, which will reduce costs of hybrid seed production for crops currently grown as hybrids and will allow crops currently grown as inbred varieties, due to prohibitively high hybrid seed production costs, to be grown as superior yielding hybrids. The disclosure concerns methods of inducing unreduced aposporous or diplosporous embryo sac formation in a sexual angiosperm comprising modifying the expression level of one or more genes.

### CROSS-REFERENCE TO RELATED APPLICATIONS

This application claims benefit and priority to an invention disclosed in U.S. Provisional Patent Application No. 63/310,092, filed Feb. 14, 2022, entitled “METHODS FOR TRANSGENICALLY INDUCING SEXUAL OR APOMICTIC REPRODUCTION”. The aforementioned application is hereby incorporated herein by reference.

### TECHNICAL FIELD

This invention relates to using asexual seed formation through gametophytic apomixis in plants to simplify hybrid seed production. Specifically, it relates to methods of inducing the first step of gametophytic apomixis (genetically unreduced embryo sac formation) in sexual plants by genetic engineering.

### BACKGROUND OF THE INVENTION

This invention discloses methods of inducing genetically unreduced (2n) embryo sac (female gametophyte, megagametophyte, ES) formation in sexual angiosperms (plants hereafter) using genetic engineering to modify wild type gene expression.

### Sexual Reproduction in Flowering Plants

This invention involves modifying sexual plant reproduction such that the first step of apomixis, unreduced ES formation, occurs. These modifications are best explained using cytological photomicrographs that illustrate normal processes that occur in female ovules during sexual reproduction and how these processes are altered by apomixis to produce asexual seeds.

Sexual reproduction in angiosperms begins with meiosis, which produces genetically reduced (In) male spores (microsporogenesis) and (In) female spores (megasporogenesis). These are the immediate products of meiosis. In sexual reproduction, a single cell in the nucellus of the ovule, the megaspore mother cell (MMC), undergoes megasporogenesis to produce a haploid (In) surviving megaspore and three In spores that degenerate (FIG. 1 A, B; FIG. 2 A-C, G). Sexual microsporogenesis produces four male spores, each of which develops into a male gametophyte that contains two 1n sperm nuclei (male gametes). The surviving female megaspore is the only cell in the ovule that develops into an ES. This occurs by three rounds of endomitosis (FIG. 1 C, D). The mature ES contains the 1n egg (female gamete) and two synergid cells that collectively form the egg apparatus, two 1n polar nuclei that fuse to produce the 2n central cell, and three additional 1n cells, which do not become part of the embryo or endosperm but serve nutritional roles during seed formation (FIG. 1 E). Sexual reproduction concludes with double fertilization, where one of the two sperm nuclei of a pollen grain fuses with the egg (syngamy) to produce the 2n zygote, which develops by mitosis into an embryo and seedling, and the other sperm nucleus fuses with the central cell to produce the 3n endosperm [1].

### Apomixis in Flowering Plants

Apomixis involves three steps: i) formation of an unreduced ES that is genetically identical to the mother plant, ii) development of a clonal embryo from the unreduced egg of the ES by parthenogenesis (without fertilization), and iii) formation of endosperm from the central cell of the ES, which supplies nutrients to the embryo as it grows and develops into a seedling. Certain species from 195 plant genera (ca. 1.5% of all genera) [2] exhibit one of three asexual developmental alternatives that cause apomixis. The most common of these is apospory, which occurs in species from ca. 130 of 13,000 angiospermous genera (1%) [2, 3]. Here, the sexual process fails during megasporogenesis or shortly thereafter such that all spores degenerate. In their place, one and sometimes up to a few unreduced (2n) ESs form from adjacent cell(s) of the ovule wall (FIG. 2 F, H-K; [4]). The next most common form of apomixis in plants is Taraxacum type diplospory [2, 3]. Here, the first division (reductional) of meiosis fails. This is followed by the second mitotic-like division of meiosis to produce two unreduced (2n) spores, of which one degenerates and one produces the unreduced ES (FIG. 2 D, E, [4]). The least common type of apomixis in plants involves a complete absence of meiosis. In these cases, the unreduced ES forms directly from the MMC. This is called Antennaria type diplospory or gonial apospory (FIG. 2 L, M; [4]) [2, 3]. Unreduced eggs in ESs of apomicts develop parthenogenetically into an embryo and seedling, and the endosperm develops from the central cell either pseudogamously (following fertilization of the central cell) or autonomously (without fertilization of the central cell). All three types of gametophytic apomixis can be obligate, meaning that all seeds of a plant form apomictically, or facultative, meaning that some seeds on a single plant form apomictically while others form sexually [1, 3, 5]. Sporophytic apomixis (or nucellar embryony) is the other major type of asexual seed formation. It differs from gametophytic apomixis in that the clonal embryo forms directly from a somatic cell of the ovule wall, usually a nucellar cell, rather than from the unfertilized egg in an unreduced ES [1, 3, 5]. When used hereafter in the present invention, the term apomixis refers to gametophytic apomixis.

### Converting Sexual Crops to Apomixis

The present invention provides methods for inducing in sexual plants 2n ES formation of the apospory type based on four defining characteristics of aposporous ESs (AES) [3, 5-8]: i) they form from cells of the nucellus or integuments (cells of the ovule wall), ii) they usually form adjacent to the MMC, degenerating sexual tetrad, or degenerating functional megaspore or early sexual ES (SES), iii) the first stage of their development involves cell enlargement and endomitosis to produce a 2-nucleate cell, which is followed by a second and usually a third endomitosis to produce a 4-8 nucleate mature unreduced ES, and iv) the first stage of their development also includes formation of one or two large vacuoles that often form prior to the first endomitosis and constitute a visible percentage of the cell volume in cleared or sectioned cytology preparations. As diplosporous ESs of the natural Taraxacum type and the natural Antennaria type are occasionally induced in addition to aposporous ESs, the present invention provides methods of inducing these types of apomictic ESs in sexual plants as well. Prior to becoming multinucleate and vacuolate, large nucellar cells are appropriately referred to simply as large nucellar cells. These are sometimes referred to as aposporous initials (AIs) if they meet a reasonable size standard, e.g., as large as or larger than the sexual meiocyte, tetrad, enlarging functional megaspore or early multinucleate and vacuolate SES [9]. Methods of inducing multinucleate and vacuolate AESs, which meet all four AES criteria (above), are found in the detailed Examples below. For comparison purposes, images of AESs that meet these standards, as observed cytologically in naturally occurring aposporous apomicts, are provided in FIG. 2 (F, H-K) [4].

Expression levels of hundreds of genes have been correlated with apospory. However, none of these genes to date have been shown to induce vacuolate multinucleate AES formation in sexual plants when their expression is up or down regulated by genetic engineering [10, 11]. Some studies claim such induction. However, photomicrographs accompanying these studies sometimes show large nucellar cells next to tetrads that are as large as or larger than the MMC, meiocyte or full tetrad, but none of these nucellar cells are vacuolate or multinucleate. Similarly large AI-like cells, which never develop into AESs, are often observed in sexual plants, as shown below for wild type Arabidopsis thaliana and as shown in [5, 6]. The present invention discloses methods of inducing AESs that meet all four criteria as listed above. All other published studies to date that claim AES induction in sexual plants by genetic engineering fail to meet the defining third and fourth criteria above, i.e., AESs that begin as enlarged nucellar cells that produce two nuclei and one or more large vacuoles. Capturing such early stage multinucleate and vacuolate AESs next to degenerating sexual megaspores in the same photomicrograph is the easiest and most definitive proof that apospory has been induced. Yet such images are missing in the examples of putatively induced apospory as cited in the following two paragraphs.

The first report of apospory being induced by genetic engineering was published in 2010. It involved down-regulating ARGONAUTE 9 (AGO9), SUPPRESSOR OF GENE SILENCING 3, and RNA-DEPENDENT RNA POLYMERASE 6, and it claimed high frequency formation of enlarged nucellar cells as well as mature multinucleate AESs in the genetically engineered A. thaliana (43-44% of ovules) [12]. Photomicrographs from this study do display large nucellar cells forming next to sexual tetrads or early SESs. However, these nucellar cells were not multinucleate, none contained a large vacuole, and none of them were obviously replacing SESs. Only one nucellar cell, which was non-vacuolate and single-nucleate, was as large as the tetrad, which reasonably qualifies it as an AI but not as an AES (FIG. 2 k; in [12]). Two images of later stage maturing SESs were presented to represent high frequency multiple AES formation (FIG. 2 o, p; in [12]). However, these images are typical of 4-8 nucleate kidney bean shaped SESs with a large vacuole separating nuclei destined to become the egg apparatus at the micropylar end from nuclei destined to become antipodals at the chalazal end. This is the typical shape of single SESs in A. thaliana at this stage of development [13, 14]. As emphasized above, the easiest to obtain cytological evidence of apospory, and the most conclusive, are photomicrographs of early stage vacuolate, multinucleate AESs that are replacing the degenerating tetrad, including its functional megaspore or young SES. Absence of such evidence in the AGO9 report [12] coupled with absence of subsequent literature extending the research concerning the reported apospory casts doubt on the photomicrograph interpretations in this early report.

Conspicuously absent in all other reports of genetically engineered induction of apospory or apospory like development in sexual plants are photomicrographs showing young, vacuolate and multi-nucleate AESs replacing early stage SESs [14-17]. Like the AGO9 report [12], these reports show either i) enlarged, single-nucleate and non-vacuolate nucellar cells that are not replacing the sexually derived ES and/or ii) later stage maturing SESs that are misinterpreted as multiple AESs. Absence of easy to obtain cytological proof of apospory replacing sexual development (if it is occurring) plus absence of follow-on studies that verify apospory and advance the initial findings casts doubt on the claimed apospory presented in these studies.

The present invention discloses wild type genes that upon their down regulation unequivocally induce AES formation in sexual plants. Photomicrographs of apospory in the down regulated germplasm meet all four criteria. The identified genes and methods may be used as part of a genetic engineering strategy to convert sexual crops to apomixis, which will reduce costs of hybrid seed production for crops currently grown as hybrids and will allow crops currently grown as inbred varieties, due to prohibitively high hybrid seed production costs, to be grown as superior yielding hybrids.

While the benefits of an apomixis technology have been recognized for at least 80 years, progress toward achieving the necessary breakthroughs has been painfully slow [1, 3, 18-22]. This sluggishness in harnessing apomixis for hybrid seed production may be a consequence of misconceptions concerning how apomixis evolved and how it is regulated. For 118 years, it has been taught and considered obvious that apomixis in plants and animals evolves secondarily and polyphyletically from sexual mechanisms through de novo mutations that have occurred many times in thousands of different lineages [5, 23-26]. The putative mutations are thought to have caused apomixis directly (mutation-derived modifications of meiosis and syngamy that convert these processes to apomeiosis and parthenogenesis, respectively) [6, 27, 28] or to distort sexual processes epigenetically to bypass certain steps of meiosis and syngamy [1, 3, 22, 25, 26, 29]. Research programs, focused for more than 60 years on Mendelian analyses of apomixis, concluded that apomixis is simply inherited [28, 30]. More recent research has focused on genetically mapping the putative apomixis-causing mutations in naturally-occurring apomicts and to then transfer the causal mutations to important world crops by wide hybridization or genetic engineering. Most recently, a major goal has been to mutate meiosis genes to create a synthetic apomixis that mimics natural apomixis [1, 25]. This “synthetic apomixis” approach, has achieved encouraging results in recent years [31-33].

### Plant Breeding Approaches With and Without Apomixis

With apomixis, plant breeding in the 21^{st} century will not be restrained by 20^{th} century inbred line requirements, but will be freed to explore previously uncontemplated yield potentials. The vision here is that sexual reproduction will be used to produce, by any type of plant breeding method, the highest yielding individual hybrid plants possible, i.e., approaching their unrestrained genetic yield potentials. Clonal seeds of these superior hybrids will then be economically produced by apomixis in commercial quantities for farmers. Expected yield gains for inbred crops such as wheat, soybean, cotton and rice, based on conventional hybrid breeding practices, range from 15-30% above the best inbred lines used as crops today [34]. By removing the inbred parent line requirement for producing uniform hybrid seed, the genetic yield ceiling might possibly surpass these levels by an additional 10-15%.

### The Synthetic Apomixis Approach for Achieving Apomixis in Crops

The “synthetic apomixis” approach to date employs three mutations, inactivation of a fourth gene, and ectopic expression of a fifth gene. The three mutated genes and the inactivated gene prevent meiotic chromosome pairing and recombination (during meiotic prophase), prevent migration of sister chromatids to poles during meiosis I, and eliminate meiosis II such that replicated sister chromatids separate during a single mitotic like division (MiMe, or Mitosis instead of Meiosis) [35, 36]. An unreduced ES then forms from a product of this aborted meiosis. Synthetic apomixis as currently developed resembles Taraxacum type diplosporous apomixis, i.e., two unreduced spores form following a meiotic 1^{st} division restitution (FIG. 2 D, E, [4]). This process is then combined with a variety of approaches for inducing parthenogenesis from the unreduced egg of the unreduced ES. The most successful method to date involves ectopically expressing the BABYBOOM (BBM) AP2 transcription factor in the unreduced egg [31, 37]. Ectopic expression of BBMusing an egg specific promotor induces parthenogenesis whether the BBM gene is derived from a naturally apomictic plant or from a sexual plant [31, 38]. In the best reported cases to date, 84% and 95% of seeds that formed were clonal for an engineered varietal rice and an engineered hybrid rice, respectively. However, percentage seed set (fertility) for these plants was reduced, being only 74% and 36%, respectively. Incomplete penetrance of misregulated or mutated genes and genomic aberrations associated with disturbed cell divisions were cited as possible causes of the reduced fertility [31].

### The Natural Apomixis Approach for Achieving Apomixis in Crops

Essentially complete seed fill (fertility) and genetic uniformity is often observed in naturally occurring apomictic plants [5, 21, 39, 40]. Accordingly, the methods disclosed herein for inducing unreduced ES formation, as observed in apomictic plants that occur in nature, may become preferred over those of the synthetic apomixis approach.

Evidence is accumulating that sexual and apomictic reproduction are polyphenic in plants [41] and other eukaryotes [9, 41, 42]. Polyphenic traits (either/or development) are encoded by a single genome [43]. Environmental cell signaling regulates which trait is expressed, and switching from one to another involves metabolism driven modifications of enzyme function and epigenetic modification of gene expression [43-47]. Examples include caterpillars changing into butterflies, tadpoles changing into frogs, and vegetative plants changing into flowering plants. Altered metabolic and epigenomic states induce the formation of altered phenotypes, which are better suited for the environmental parameters that induced their onset. The present disclosure provides methods that epigenetically suppress meiosis and SES formation, rather than mutating these processes, while epigenetically promoting unreduced ES formation directly from unreduced somatic cells of the ovule wall, i.e., apospory, or from a 1^{st} division restitution meiocyte, i.e., Taraxacum type diplospory, or from a completely ameiotic MMC, i.e., Antennaria type diplospory.

### Spontaneous Haploid Genome Doubling

In addition to enabling clonal (apomictic) seed set of hybrid crop plants, the present disclosure of the invention can be used to produce unreduced, and hence fertile, ESs in haploid plants, which are typically sterile due to gross genetic imbalances resulting from attempted meiotic reductions of single (haploid) genomes. In the spontaneous haploid genome doubling approach to plant breeding, haploid plants are produced by various methods including direct formation from immature pollen grains in another culture. When the donor pollen grains are obtained from an Fi hybrid plant, the haploid plants produced represent a myriad of genetically heterogeneous genotypes some of which may have unique gene combinations that are of potentially high value when used as parent lines for producing superior yielding crop hybrids. By avoiding meiosis via production of AESs (per the present invention), fully functional (fertile) haploid female eggs are then available to be fertilized by genetically identical unreduced haploid sperm, which often form on the same plant in sufficient quantities for fertilization. The plants that result are instantaneously homozygous, and these can then be tested for their ability to produce high yielding hybrids when crossed with other lines (FIG. 3). This is an example of doubled haploid technology, which is currently used to accelerate the production of parent lines in hybrid breeding programs such as those used for producing hybrids of maize [48]. The use of an unreduced AES technology as presented in the present disclosure could greatly accelerate homozygous parent line production in hybrid breeding programs of hybrid crops.

### SUMMARY OF THE INVENTION

The present disclosure in aspects and embodiments addresses the various needs and problems of introducing apomixis technology into plant breeding. It is based on a recent hypothesis that many if not most eukaryotes have retained in their genomes an ancient polyphenism (capacity) for expressing both modes of reproduction such that misregulating certain wild type genes will initiate the essential “single cell” developmental phenomena for either apomixis (unreduced egg formation and parthenogenesis) or sex (meiosis and syngamy) [9, 41, 42].

In some aspects, the disclosure concerns methods of inducing unreduced aposporous or diplosporous ES formation in a sexual angiosperm comprising up regulation of the abscisic acid (ABA) signal transduction pathway. In some methods, up regulation of ABA signaling comprises reducing the expression level of one or more genes that normally suppress ABA signaling selected from PPRT1, AITR1, USB1, CYP707a3 and CYP07a1 and orthologs thereof.

In some aspects, the disclosure concerns methods of inducing unreduced aposporous or diplosporous ES formation in a sexual angiosperm comprising modifying the SnRK2 signal transduction pathway. In some methods, modifying SnRK signaling comprises reducing the expression level of SnRK2.9 and orthologs thereof.

In some aspects, the disclosure concerns methods of inducing unreduced aposporous or diplosporous ES formation in a sexual angiosperm comprising modifying photosynthesis gene expression.

In some methods, modifying photosynthesis gene expression comprises modifying the expression level of NAC060 and orthologs thereof.

Some aspects concern methods of inducing aposporous or diplosporous ES formation in a sexual angiosperm comprising regulation of the brassinosteroid signal transduction pathway. In some embodiments, up regulation of brassinosteroid signaling involves reducing the expression level of BIN2 and orthologs thereof.

Certain aspects concern methods of inducing aposporous or diplosporous ES formation in a sexual angiosperm comprising regulation of oxidative stress attenuation pathways. In some embodiments, the regulation of oxidative stress attenuation pathways involves modifying the expression of TG (RAP2.4a) and orthologs thereof.

Another aspect of the disclosure concerns methods of inducing unreduced aposporous or diplosporous ES formation in a sexual angiosperm comprising modifying the expression level of one or more genes selected from PPRT1, AITR1, USBI-1, CYP707a3, CYP07a1, TG, SNRK2.9, NAC060, BIN2 and orthologs thereof.

In some embodiments, modifying the expression level of the one or more genes reduces the expression of the gene. In certain embodiments modifying the expression level of the one or more genes knocks out expression of the gene.

Some methods are a step in inducing apomixis.

In certain embodiments, the sexual angiosperm is selected from: alfalfa, amaranth, asparagus, barley, beans, beets, buckwheat, canary grass, cacao, carob, carrots, castor beans, chickpeas, chilis, clover, coffee, cotton, cowpea, cucumbers, cucurbits, durum, flax (linseed), fonio, Job's tears, kaniwa, lentils, lettuce, lupin beans, maize (corn), melons, mesquite, millet, oat, onions, peanuts, peas, peppers, pitseed goosefoot, quinoa, rapeseed, rice, rye, sorghum, soybean, spelt, squash, sunflower, tamarind, teff, tomato, triticale, turnips, wheat, and wild rice.

In some embodiments, the sexual angiosperm plant is a member of the Brassicaceae, Fabaceae, Asteraceae, or Poaceae family.

In certain embodiments the methods are used in polyploid reproduction. Some polyploids are a triploid such as a banana plant.

In some embodiments, the sexual angiosperm is part of a rice, corn, or wheat plant. In other embodiments, the sexual angiosperm is part of a rose, petunia or lily plant. Yet other embodiments concern broccoli, kale, eggplant, tomato, pepper or sugarcane plant.

In certain embodiments, the method induces at least 5% or at least 25% or at least 50% or at least 75% or at least 90% unreduced aposporous or diplosporous ES formation in the sexual angiosperm.

Some aspects concern methods of inducing apomixis in flowing plants comprising: formation of an unreduced ES that is genetically identical to the mother plant, the formation comprising inducing unreduced aposporous or diplosporous ES formation by modifying the expression level of one or more genes selected from PPRT1, AITR1, USBI-1, CYP707a3, CYP07a1, TG, SNRK2.9, NAC060, BIN2 and orthologs; development of a clonal embryo from the unreduced egg of the ES by parthenogenesis; and formation of endosperm from the central cell of the ES, which supplies nutrients to the embryo as it grows and develops into a seedling.

Further aspects concern methods of converting sexual crops to apomixis comprising inducing unreduced aposporous or diplosporous ES formation by modifying the expression level of one or more genes selected from PPRT1, AITR1, USBI-1, CYP707a3, CYP07a1, TG, SNRK2.9, NAC060, BIN2 and orthologs.

In some embodiments, the disclosure provides a plant variety that produces unreduced AESs according to the methods set forth herein.

In some embodiments, the disclosure provides methods for inducing unreduced ES formation such that methods for inducing parthenogenesis may be combined with it to efficiently clone superior genotypes of important crop species by gametophytic apomixis.

In some embodiments, the disclosure provides methods for producing functional unreduced ESs in haploid plants, often derived by another culture, to accelerate the production of doubled haploid parent lines in hybrid breeding programs of maize and other crops.

### BRIEF DESCRIPTION OF THE FIGURES

FIG. 1. Presents representative images of sexual megasporogenesis (female meiosis) and ES (female gametophyte) development in wild type A. thaliana. (A) Young sexual ovule in which a MMC has formed at the micropylar end of the nucellus. The integuments appear only as buds at the base of the nucellus. (B) A slightly older sexual ovule in which the MMC has completed the two divisions of meiosis to produce a tetrad with a surviving megaspore, which will produce the ES, and three smaller megaspores in the early stages of degeneration. (C) A slightly more mature sexual ovule with a vacuolate 2-nucleate ES, which is the next stage of normal ES formation in angiosperms [8]. (D) An older sexual ovule at the 4-nucleate stage, the nucellus is completely degenerated as are some of the nucellar epidermis cells. (E) A sexual ovule containing a strongly vacuolate maturing ES that is nearing the stage of fertilization. The egg apparatus, consisting of two synergids and the egg, and a polar nucleus are visible. 2es, 2-nucleate SES; 4es, 4-nucleate SES; ch-e, chalazal end; ds, degenerating spore; ea, egg apparatus; i, integument; m-e, micropylar end; mes, mature SES; MMC, megaspore mother cell; n, nucleus; nc, nucellar cell; ne, nucellar epidermis; pn, polar nucleus; ss, surviving spore.

FIG. 2 presents representative images of sexual megasporogenesis and Taraxacum-type diplosporous, Hieracium-type aposporous, and Antennaria-type diplosporous (gonial apospory) ES development in Boechera (Brassicaceae). (A) Row of three adjacent ovules in the genetically reduced tetrad stage of meiosis in the sexual species B. stricta. (B) Sexual tetrad in a B. yellowstonensis ovule. (C) Sexual tetrad in a B. exilis ovule. (D) Unreduced Taraxacum-type dyad in a B. exilis×thompsonii ovule (=B. lignifera). (E) Unreduced Taraxacum-type dyad in a B. crandalli×gracilipes ovule. (F) Unreduced 1-nucleate aposporous ES with degenerating unreduced Taraxacum-type dyad in a B. retrofracta×stricta ovule. (G) Sexual MMC with a parietal cell in a B. yellowstonensis ovule. (H) Unreduced 2-nucleate aposporous ES with a degenerating sexual tetrad in a B. cusickii×sparsiflora ovule. (I) Four unreduced 2-nucleate aposporous ES in a B. imnahaensis×yellowstonensis ovule (=B. microphylla). (J) Two unreduced 1-nucleate Hieracium-type aposporous ES, an AI, and a degenerating tetrad in a B. retrofracta×stricta ovule. (K) Unreduced 2-nucleate and unreduced 4-nucleate AES in a B. crandallii×thompsonii ovule (=B. pallidifolia). (L-M) Unreduced 1-nucleate diplosporous ES forming directly from the MMC in a B. retrofracta×stricta ovule. Thick black arrows, degenerating megaspores; white arrows, surviving megaspores; ai, aposporous initial cell; ii, inner integument; oi, outer integument; p, parietal cell; v, vacuole; bars, 20 μm. (from [4]).

FIG. 3. Shows enhancing sexual fertility of maize haploids by inducing unreduced functional male and female gametophyte formation.

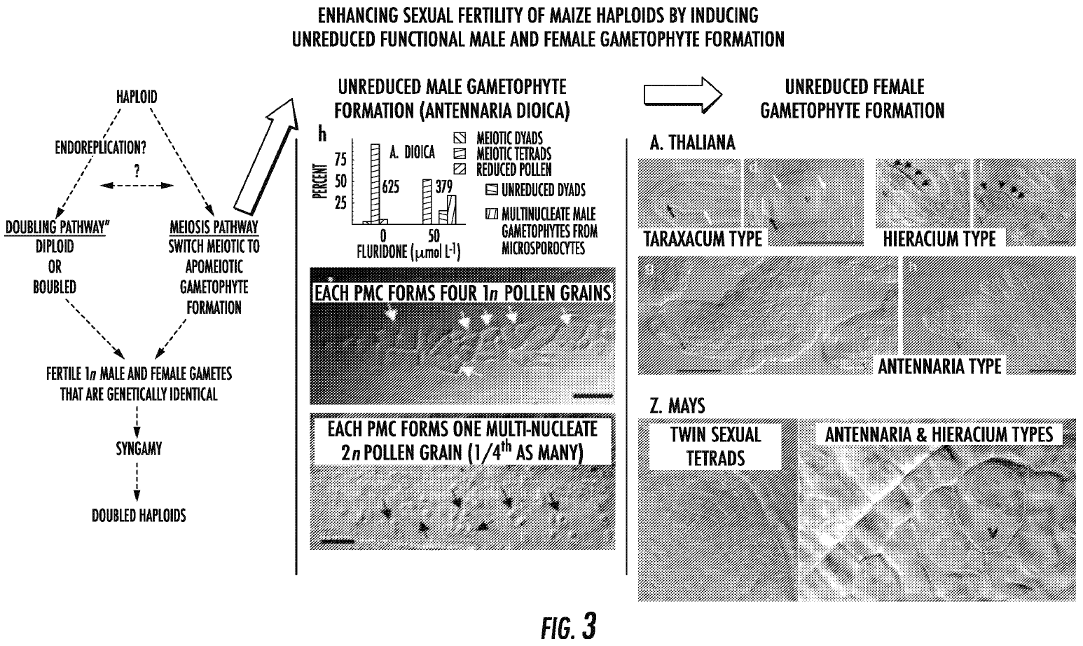

FIG. 4 presents candidate knock-out gene details—AITR1-2.

FIG. 5 presents candidate knock-out gene details—BIN2-2.

FIG. 6 presents candidate knock-out gene details—CYP707A1-1.

FIG. 7 presents candidate knock-out gene details—CYP707A1-2.

FIG. 8 presents candidate knock-out gene details—CYP707A3-1.

FIG. 9 presents candidate knock-out gene details—CYP707A3-2.

FIG. 10 presents candidate knock-out gene details—NAC060-1.

FIG. 11 presents candidate knock-out gene details—PPRT1-1.

FIG. 12 presents candidate knock-out gene details—SnRK2.9-1.

FIG. 13 presents candidate knock-out gene details—SnRK2.9-2.

FIG. 14 presents candidate knock-out gene details—TG, WIND3-1.

FIG. 15 presents candidate knock-out gene details—USB1-1.

FIG. 16 shows DNA gel depicting DNA from seven putative cyp707a3 knock-out plants. One homozygous knock-out plant (lanes 6 and 7, used for cytological analyses), two heterozygous plants (lanes 9-13), and four wild type plants (lanes 3 and 4, lanes 15-22) were identified.

FIG. 17 shows determining AI or AES area versus the area of sexual tetrads, functional megaspores or SESs. A, Photomicrograph of the 2-dimensional area of an aposporous initial (dashed line) versus that of the sexual functional megaspore (dotted line) in an ovule from the knock-out line cyp707a3; B, area ratios for the top, upper, lower and bottom quartiles (aposporous over sexual) of aposporous ovules from three knock-out lines, aitr2, pprt1 and cyp707a3.

FIG. 18 presents photomicrographs of AES formation in ovules of the knock-out line cyp707a3. A, vacuolate, 1-nucleate AES (aes1); B, vacuolate, 2-nucleate AES (aes2) that is replacing the four degenerating meiotic megaspores. Unlabeled arrows point to meiosis produced degenerating megaspores. Dotted lines in A denote outline of the sexual tetrad and ses1. Dashed lines in A and B denote outline of the aes1 and aes2, respectively.

FIG. 19 presents photomicrographs of AES formation in ovules of the knock-out line pprt1. A, Vacuolate, 1-nucleate AES. The three degenerating megaspores are visible as remnant tissues that are nearly completely digested. The SES is large, but in this focal plane, which favors the AES, details of its nuclei and vacuole composition are not visible. B, Vacuolate, 2-nucleate AES that is replacing three of the four degenerating meiotic megaspores. Here again, the focal plane chosen for capturing favors details of the AES. However, the size of the SES indicates that it is healthy and growing. Unlabeled arrows point to the three degenerating megaspores and the SES. Dotted lines in B denote outline of the sexual tetrad and SES. Dashed lines in B denote outline of the AES2.

FIG. 20 presents photomicrographs of AES formation in ovules of the knock-out line aitr1. A, Vacuolate, 1-nucleate AES. Two degenerating megaspores are visible as remnant tissues that are nearly completely digested. The third degenerating megaspore may have been completely digested or it may be obscured by the artifactual bright spot at the micropylar end of the nucellus. The SES1 is large with a single vacuole in focus. This focal plane, which favors the AES1, may be obscuring a second SES vacuole. B, Vacuolate, 2-nucleate AES (AES2) that is replacing three of the four degenerating meiotic megaspores. The upper AES2 nucleus contains two nucleoli, both of which are contained within a single nuclear membrane that is faintly visible and about 4× larger in area than the areas of the two nucleoli combined. The focal plane was chosen to capture details of the AES2. However, the size of the SES indicates that it is healthy and growing though not as advanced developmentally as the AES2. Unlabeled arrows point to the three degenerating megaspores and the SES. Dotted lines in A and B denote outlines of the sexual tetrads and SESs. Dashed lines in A and B denote outlines of the AESs.

FIG. 21 contains Table 1 showing potential annual value added by implementation of a fully function apomixis in 12 crops.

FIG. 22 contains Table 2 showing the percent aposporous ovules for 12 knock-out lines.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

### DETAILED DESCRIPTION OF THE INVENTION

The present disclosure covers composition and methods of transgenically inducing unreduced AES formation in sexual plants. In the following description, specific details are provided for a thorough understanding of preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the tables and figures, is not intended to limit the scope of the invention, but is merely representative of the various embodiments of the invention.

### Definitions

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

The term “In” refers to the number of chromosomes in genetically reduced spores or gametes and, in the case of spores, the number of chromosomes in subsequent mitoses that produce gametophytes and gametes (organisms with alternating generations); this number often reflects multiples of the chromosome base number x; a plus or minus followed by a small number is used to identify aneuploid additions or subtractions of one or more chromosomes from the 1n number.

The term “2n” refers to the number of chromosomes in the zygote and in subsequently produced asexual (somatic) cells that are produced mitotically; this number often reflects different ploidy levels (multiples of the base number, x) plus or minus individual chromosomes (aneuploidy).

The term “abscisic acid signaling” refers to a major stress signaling pathway particularly in land plants that, among other functions, attenuates oxidate stress by up-regulating ROS attenuation processes, and attenuates water stress by closing leaf stomata.

The term “AES” refers to an aposporous ES formed by apospory.

The term “AI” refers to an aposporous initial cell that by itself does not confirm apospory.

The term “aneuploid” refers to a plant that contains a haploid, diploid or polyploid genome plus or minus one to a few single chromosomes.

The term “apomeiosis” refers to the formation of unreduced spores, gametophytes, gametes or gamete-like cells from meiocyte mother cells, nucellar cells or integument cells wherein the chromosome numbers and genetic composition are identical to those observed in the mother plant. Apomeiosis is part of an epigenetically regulated developmental trajectory that includes parthenogenesis.

The term “apomixis” refers to a developmental trajectory in eukaryotes consisting of two temporally-distinct but epigenetically linked processes, apomeiosis and parthenogenesis.

The term “aposporous initial” (AI) refers to a non-vacuolate and single-nucleate nucellar cell that is as large as or larger than either the sexual meiocyte, the enlarged functional megaspore or the young sexual ES. Their presence by themselves does not confirm that apospory is occurring.

The term “apospory” refers to unreduced gametophyte (ES) formation (AES formation) from an unreduced cell of the sporophyte generation.

The term “autonomous apomixis” refers to seed formation in angiosperms wherein the embryo is genetically identical to the mother plant and the endosperm forms without fertilization.

The phrase “brassinosteroid signal transduction pathway” refers to brassinosteroids which are a group of steroid hormones that regulate plant growth, development and reproduction at many different levels.

The term “cyclical apomixis” refers to alternations, generally seasonal, between periods of essentially exclusive sexual reproduction and periods of essentially exclusive apomictic reproduction.

The term “DIC” refers to differential interference contrast microscopy. It is also referred to as Nomarski DIC microscopy.

The term “derepression” refers to a reversal of repression often by the chemical removal of a transcription repressor gene product from a regulatory DNA sequence.

The term “differential interference contrast” (DIC) refers to a light microscopy technique that uses additional lenses and filters so that internal structures of specimens can be visualized by optical sectioning, i.e., without physically sectioning the specimen. It is also referred to as Nomarski DIC microscopy.

The term “diploid” refers to a plant that contains a pair of genome.

The term “diplospory” refers to unreduced gametophyte formation from an unreduced cell following a 1st division restitutional division of the MMC (Taraxacum type diplospory) or directly from an ameiotic MMC (Antennaria type diplospory, also referred to as gonial apospory).

The term “double fertilization” refers to a process unique to angiosperms wherein two 1n (haploid) sperm nuclei of a single male gametophyte are involved in fertilization, one fertilizes the 1n egg, to form the 2n embryo, and the other fertilizes the 2n central cell (fusion product of two 1n polar nuclei) to form the 3n endosperm.

The term “ES” refers to the female megagametophyte (embryo sac) in angiosperms.

The term “embryo sac” refers to the female megagametophyte in angiosperms.

The term “endosperm” refers to a 3n tissue in angiospermous seeds that is adjacent to the developing angiospermous embryo and is derived in sexual plants from the fusion product of two 1n female gametophyte cells (polar nuclei) and a 1n sperm cell. Endosperm is consumed by the embryo as it grows and germinates.

The term “epigenome” refers to a genome (DNA) plus all of the chemical compounds added to the genome that regulate gene expression. In living cells, epigenome modifications are generally in a state of flux and vary among different tissues and cells.

The term “epigenomic state” refers to a static state of the genetic material wherein some genes tend to be silenced while others tend to be expressed. The epigenomic state of living organisms is in constant flux. For the purposes of this specification, we use the term epigenetic in a broad sense. Our use of the term includes covalent DNA and RNA modifications that affect gene expression such as chromatin modifications and mRNA splicing variants.

The term “eukaryogenesis” refers to an extended period of time, about 2.5 to 1.6 billion years ago, during which eukaryotes evolved from prokaryotes.

The term “eukaryote” refers to an organism whose cells contain a nucleus and other organelles enclosed by membranes.

The term “facultative apomixis” refers to sexual and apomictic reproduction occurring concurrently within an individual. It is often observed in plants that simultaneously produce seeds sexually and apomictically.

The term “gamete” refers to a mature haploid or diploid germ cell that is able to either i) unite with another gamete by syngamy (fertilization) to form a zygote, as in sexual reproduction, or ii) become a zygote-like cell by parthenogenesis, as in apomictic reproduction.

The term “gametophyte” refers to the 1n generation that produces gametes in organisms with an alternation-of-generations life cycle. In angiosperms, female megagametophytes (ESs) form in ovules and in most cases consist of seven cells (including the egg, female gamete) and eight nuclei at maturity (central cell contains two polar nuclei). Microgametophytes (pollen grains) in angiosperms generally consist of three cells, two sperm (male gametes) and a pollen tube nucleus.

The term “gametophytic apomixis” (apomixis herein) refers to a form of clonal seed formation where meiosis aborts, an unreduced gametophyte with its unreduced egg forms from one of several types of cells of the ovule, a clonal embryo forms parthenogenetically from the unreduced egg, and endosperm forms either following fertilization of the ES central cell (pseudogamous apomixis) or without central cell fertilization (autonomous apomixis). In gametophytic apomixis, the clonal embryo forms from an unreduced egg of an unreduced gametophyte, which differs from sporophytic apomixis, where the unreduced egg forms from a non-gametophyte cell of the ovule.

The term “genotype” refers to the full hereditary information of an organism. Inasmuch as progeny plants of an apomictic plant are clones of each other, they represent a single genotype.

The term “germline” refers to a series of cells destined to pass their genetic material to their progeny. In some multicellular organisms, including animals, the germline is continuous from one generation to the next. In other organisms, the germline is discontinuous. In flowering plants, the germline is initiated during flower formation and is terminated with the onset of embryogenesis.

The term “germline-associated tissues” refers to those tissues intimately associated with and thereby nutritionally and developmentally supportive of the germline. With regard to flowering plants, the term refers to the nucellus and other nutritive tissues of the ovule.

The term “haploid” refers to a plant that contains a single genome rather than a pair of genomes.

The term “Hieracium-type apospory” refers to a form of apomixis in angiosperms wherein sexual meiosis or its immediate 1n products fail and are simultaneously replaced by one or more parthenogenetically-competent 2n gametophytes that usually form from nucellar cells.

The term “meiocyte” refers to a cell undergoing meiosis.

The term “meiosis” refers to spore or gamete formation wherein chromosome numbers are reduced to half of that observed in zygotes. Meiosis is part of a single epigenetically controlled developmental trajectory that includes syngamy. If the cellular conditions that induce meiosis are strong enough and are temporally maintained, they will also enable syngamy and vice versa.

The term “megasporogenesis” refers to the formation of four haploid (In) female spores by meiosis in ovules of angiosperms.

The term “microsporogenesis” refers to the formation of four haploid (In) male spores by meiosis in anthers of angiosperms.

The term “MMC” refers to the megaspore mother cell, which in angiosperms undergoes female meiosis in the ovule to produce four genetically reduced (In) female spores.

The term “natural apomixis” refers to apomeiotic and parthenogenetic processes that occur in nature. As an approach to produce apomictic crops, it is motivated by the hypothesis that many if not most eukaryotes, whether they currently express apomixis or not, carry the potential in their genomes to express either sexual or apomictic reproduction depending on states of metabolic homeostasis.

The term “nucellus” refers to a somatic, but ancestrally sporogenous, nutritive tissue surrounding the meiocyte and later the developing ES in ovules of angiosperms.

The term “obligate apomixis” refers to reproduction by apomixis with an absence of sexual reproduction.

The term “ovule” refers to a female organ within the ovary of an angiospermous flower that becomes a seed.

The term “ovary” refers to a female organ of an angiospermous flower that becomes the seed pod of plants that produce multiple ovules per ovary or the outer coating of seeds that produce a single ovule per ovary.

The phrase “oxidative stress” refers to an imbalance between the production and accumulation of reactive oxygen species (ROS) in cells or tissues and the ability of these cells or tissues to detoxify them, Oxidative stress functions as a second messenger that post-translationally modifies proteins that in turn induce the up-regulation of ROS attenuation mechanisms.

The term “parthenogenesis” refers to life cycle progression without syngamy from products of either meiosis or apomeiosis, e.g., embryo formation from reduced or unreduced eggs without fertilization.

Parthenogenesis from products of meiosis is referred to as haploid parthenogenesis.

Parthenogenesis is part of a single epigenetically controlled developmental trajectory that includes apomeiosis. If the cellular conditions that induce parthenogenesis are strong enough and are temporally maintained, they will also induce apomeiosis and vice versa.

The term “phenism” refers to one of two or more phenotypes expressed polyphenically by a single genotype.

The term “phenotype” refers to observable characteristics of an individual genotype as a result of environment-genotype interactions.

The term “polyphenism” refers to a set of two or more phenotypes (phenisms) that occur in a single individual but at different times or under different environmental conditions. Classic examples include metamorphism in insects, the transition from a tadpole to a frog, or the transition from a vegetative to a reproductive state in plants. Chromatin modifications that differentially silence genes, thus producing different epigenomic states, are responsible for polyphenisms.

The term “polyphyletic trait” refers to a trait that occurs in multiple taxa but evolved in each taxon independently of the other taxa.

The term “polyploid” refers to a plant that contains three or more complete sets (genomes) of chromosomes.

The term “preapomeiotic” refers to the stage of apomictic reproduction wherein meiocyte mother cells and associated cells and tissues are not yet mature or are still forming.

The term “premeiotic” refers to the stage of sexual reproduction wherein meiocyte mother cells are not yet mature or are still forming.

The term “prokaryote” refers to single-celled archaea and bacteria. They do not possess a distinct membrane-bound nucleus, mitochondria or other specialized organelles.

The term “pseudogamous apomixis” refers to seed formation in angiosperms wherein the embryo is genetically identical to the mother plant and the central cell of the ES must be fertilized for endosperm to form. Without fertilization, seeds of pseudogamous apomicts usually abort.

The term “reactive oxygen species” (ROS) refers to unstable oxygen containing molecules, e.g., H₂O₂, that react with other molecules in cells potentially damaging them as well as inducing cell signaling processes that up-regulate ROS attenuation mechanisms.

The term “repression” refers to suppression of gene expression, which often occurs as a result of a repressor gene product that blocks transcription.

The term “SES” refers to a sexual ES formed following female meiosis in angiosperms.

The term “sexual reproduction (sex)” refers to a process in eukaryotes consisting of two temporally-distinct but epigenetically linked processes, meiosis and syngamy (fertilization).

The term “signaling molecule” refers to a molecule that interacts with cell surface receptors or otherwise participates as a component of a signal transduction pathway.

The term “SnRK2” refers to genes that play fundamental roles in energy and stress signaling pathways that regulate gene expression.

The term “sporophyte” refers to the 2n generation in organisms with an alternation-of-generations life cycle. In sexual angiosperms, sporophytes produce 1n spores through meiosis.

The term “sporophytic apomixis” refers to embryo formation from a somatic cell of the sporophyte, e.g., from a nucellar cell of the angiosperm ovule. In angiosperms, this form of apomixis is often accompanied by sexual gametophyte formation followed by functional fertilization-dependent endosperm formation from the gametophyte central cell, which nourishes the sporophyte derived somatic embryo.

The term “steroid” refers to a class of organic molecules that contain a characteristic four-ring configuration. Many steroids function as signaling molecules by activating steroid receptors.

The term “syngamy” refers to fusion of haploid gametes to produce zygotes. Syngamy is part of a single epigenetically controlled developmental trajectory that includes meiosis. If the cellular conditions that induce syngamy are strong enough and are temporally maintained, they will also induce meiosis, and vice versa.

The term “synthetic apomixis” refers to genetic engineering processes that mutate specific meiosis and/or syngamy functions the results of which mimic natural apomixis. As an approach to produce apomictic crops, it is motivated by the hypothesis that apomixis arises in sexual eukaryotes by mutation.

The term “Taraxacum-type diplospory” refers to a form of apomixis in angiosperms wherein the unreduced spore forms as a result of 1st division restitution; 2n gametophyte formation and parthenogenesis then ensue.

The term “x” refers to the “base number” of chromosomes; it constitutes the lowest number of chromosomes that represent one complete set of chromosomes for a species.

The term “zygote” refers to a cell resulting from the fusion of two gametes.

### Value of the Methods of the Invention

The present invention provides methods for avoiding both meiosis (genetic reduction) and reduced (In) ES formation by developmentally replacing both of these sexual processes with unreduced (2n) AES formation. This invention provides a natural and efficient alternative to the current practice of mutating meiosis to obtain unreduced spores that subsequently produce unreduced ESs [31].

Enabling clonal seed formation by the present invention may occur independently of other technologies, e.g., by enhancing current apospory inducing signals by simultaneously downregulating two or more of the disclosed genes, or in combination with existing technologies, such as upregulating either the BABYBOOM gene [31] or the PAR gene [38] using an egg specific promotor so as to induce parthenogenesis in a plant modified by the present invention to produce unreduced AESs.

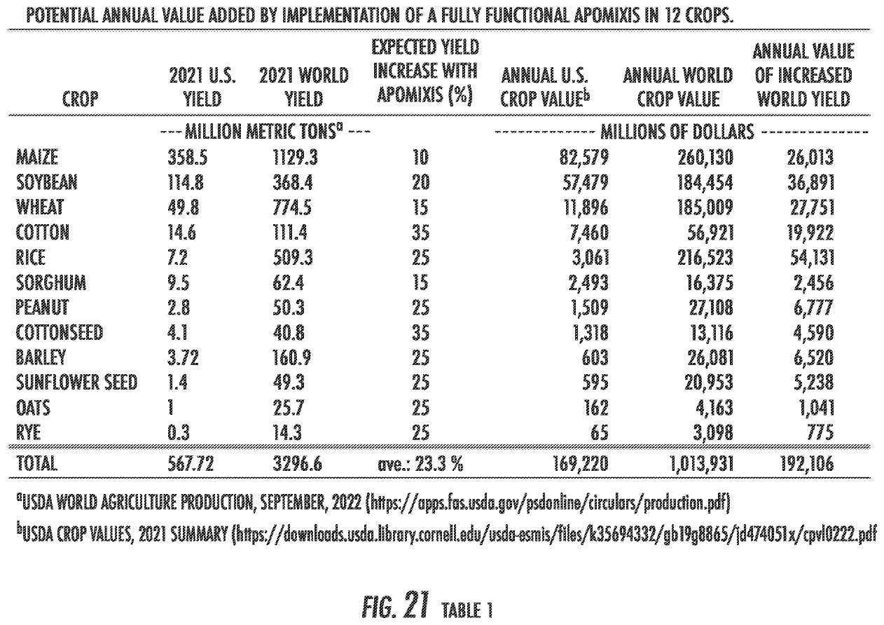

One of several important uses of the present invention is to stably clone high-yielding heterozygous hybrids or other superior genotypes of crops through their own seed. In this way, the invention by itself or in combination with existing technologies will enhance global food security. It will:
i. simplify hybrid seed production for crops currently grown as hybrids, e.g., a cost savings is expected of ca. $0.8 billion annually in producing hybrid corn seed in the U.S. alone;
ii. enable the conversion of inbred (or varietal) crops to hybrid crops. Essentially all world wheat and soybean production and most rice production today is obtained from inbred varieties. Inbred varieties of wheat and rice currently provide ca. 65% of all calories consumed by humans. Existing experimental hybrids of wheat and rice yield ca. 15% and 30% more grain, respectively [34], enough to provide sufficient calories for an additional billion people. The value of the increased yield afforded by apomictically produced hybrids of wheat and rice in the U.S. alone is $34 billion annually based on 2010-2015 USDA statistics. The value of an average 23.3% increase in yield from apomictic hybrids of 12 inbred world crops is $192 billion annually (TABLE 1, FIG. 21).

The current disclosure is not limited to agricultural crops. It may also be used, for example, to produce high yielding apomicts for the timber and fiber (flax, cotton, hemp) industries. This technology, once fully implemented, has the potential of revolutionizing food, feed, fiber and timber production globally and producing economic and humanitarian benefits that dwarf those associated with the development of sexually-produced hybrid crops in the 1930s or the green revolution of the 1960s [49].

A further use of the present invention is to enable high frequency production of unreduced and fertile egg-containing ESs in haploid plants, which normally are sterile. This use of the invention, i.e., to spontaneously produce doubled haploids from haploid plants, could greatly improve the efficiency of producing potentially valuable homozygous parent lines in hybrid breeding programs.

### Summary of Experiments

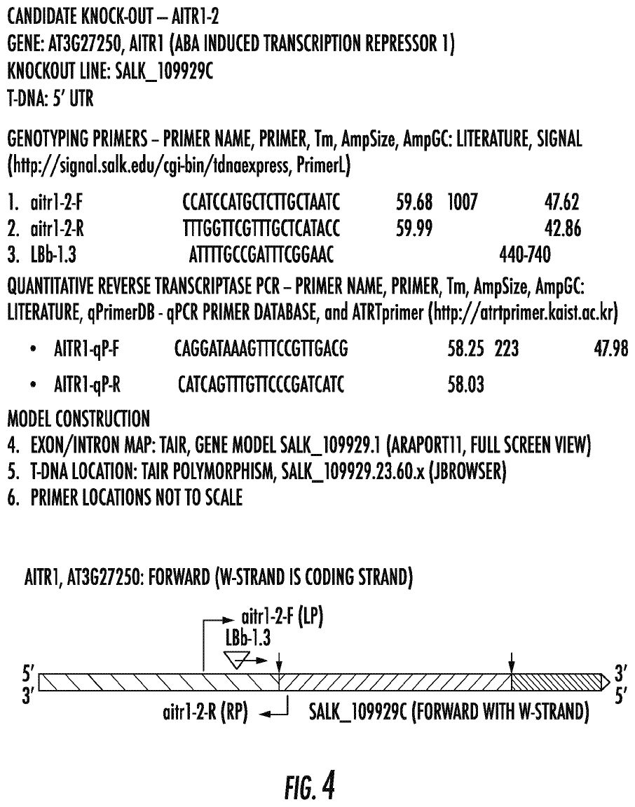

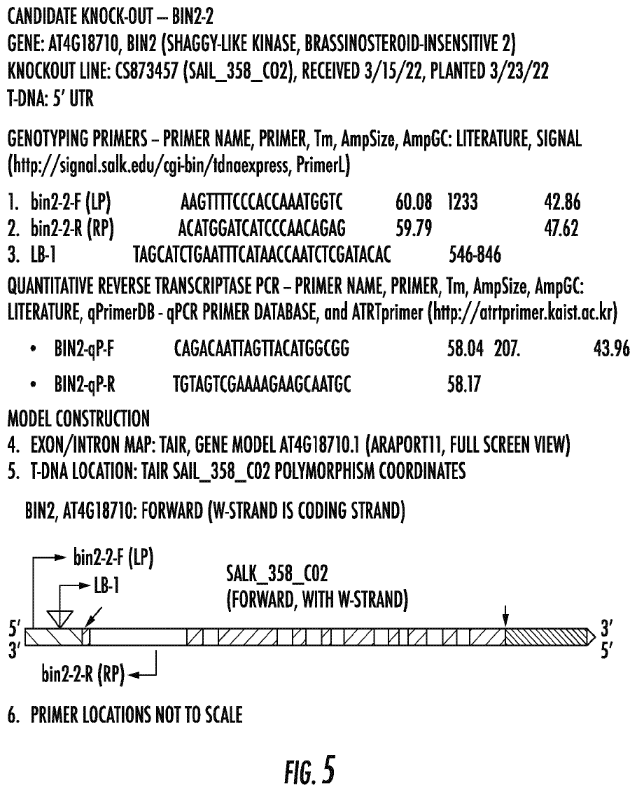

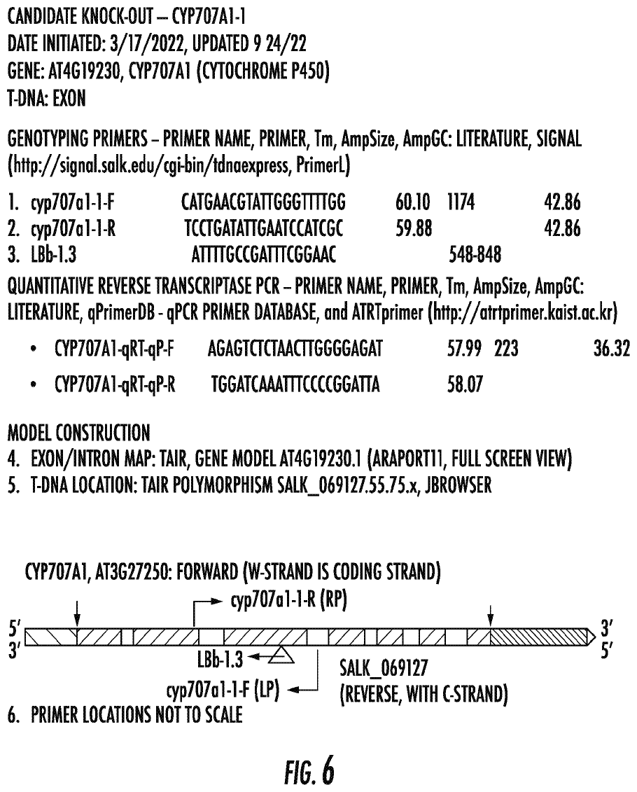

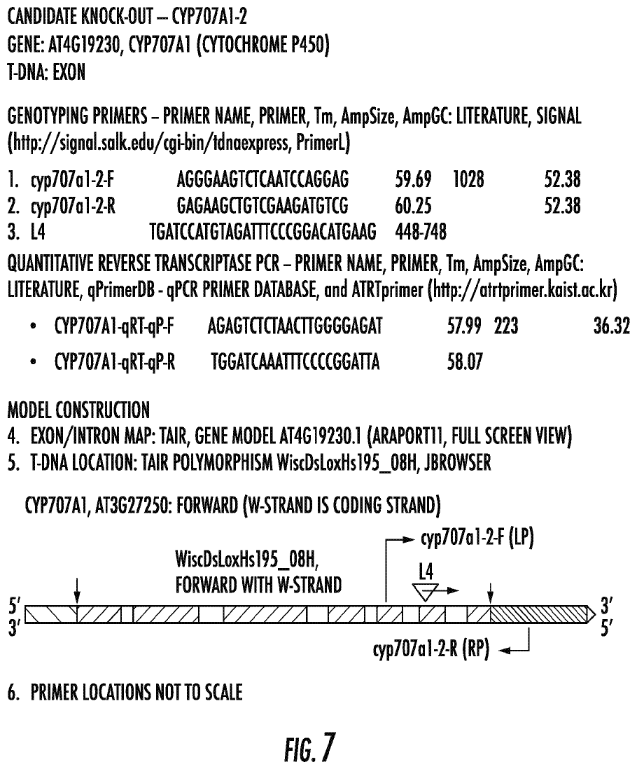

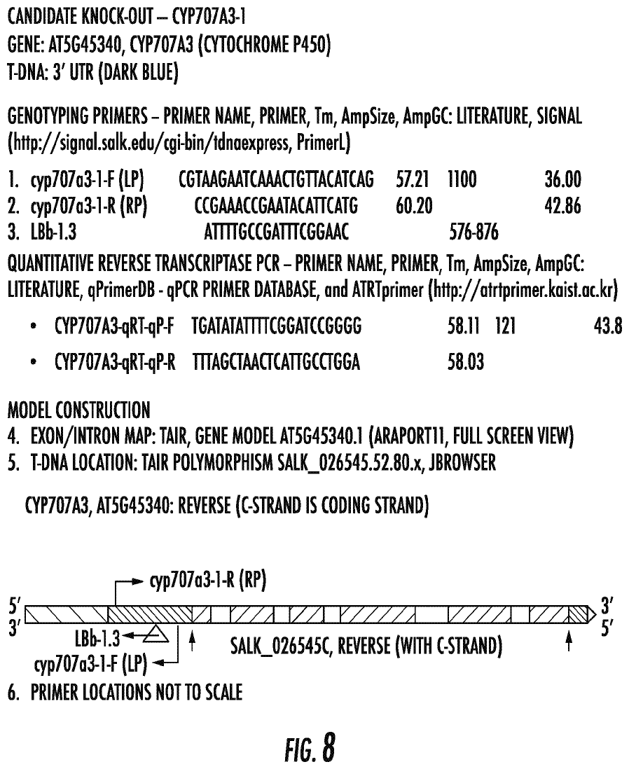

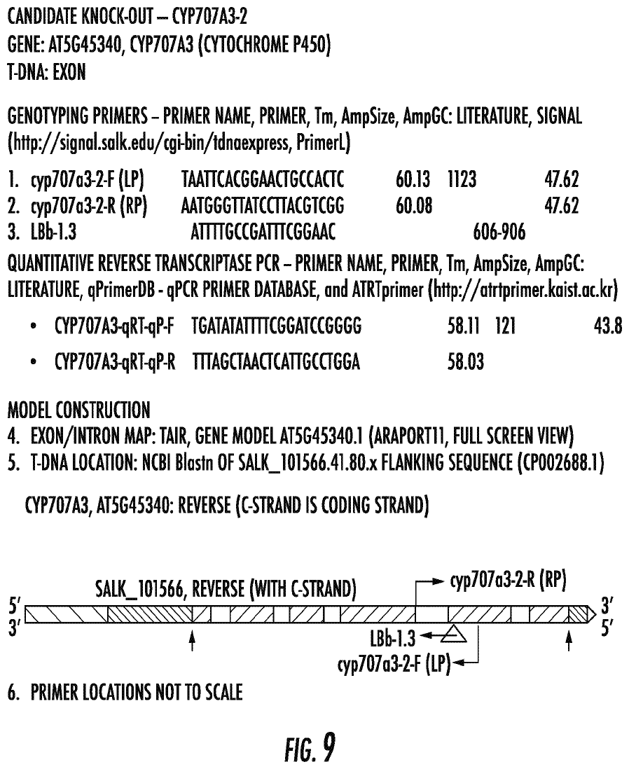

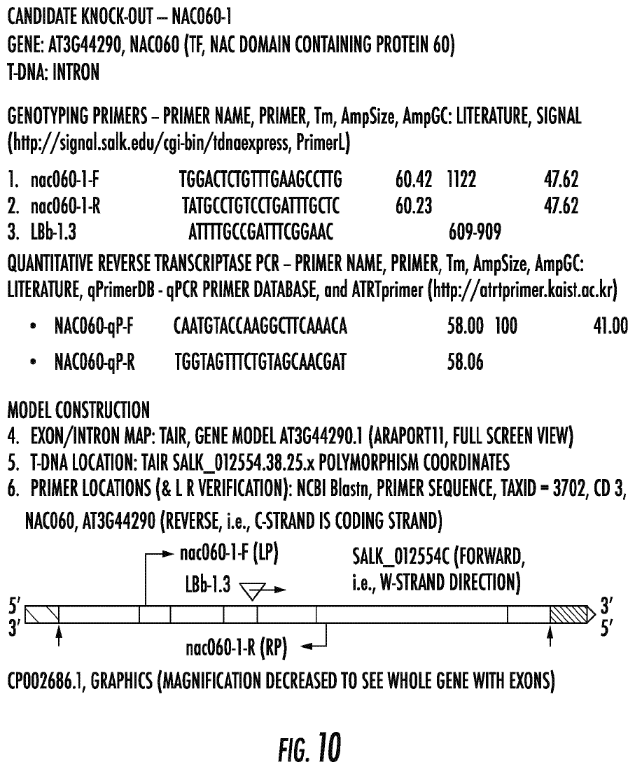

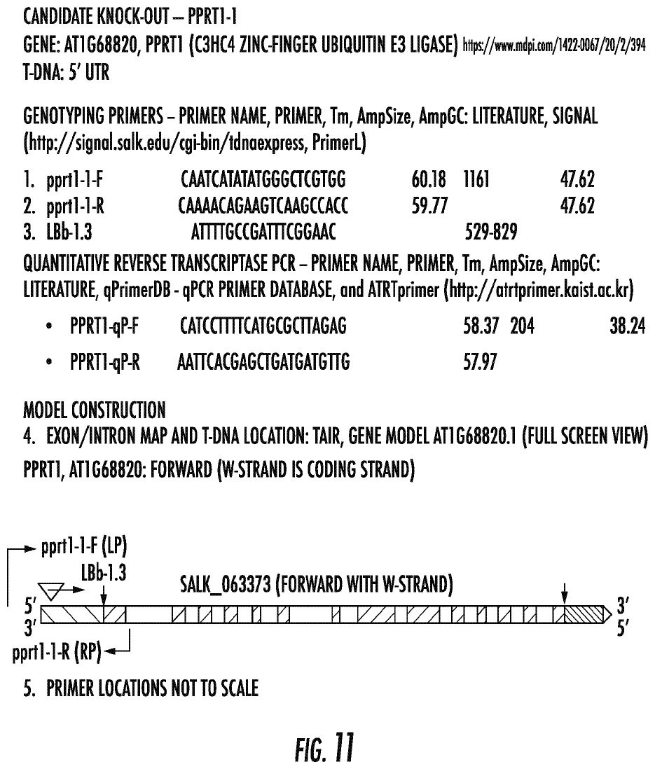

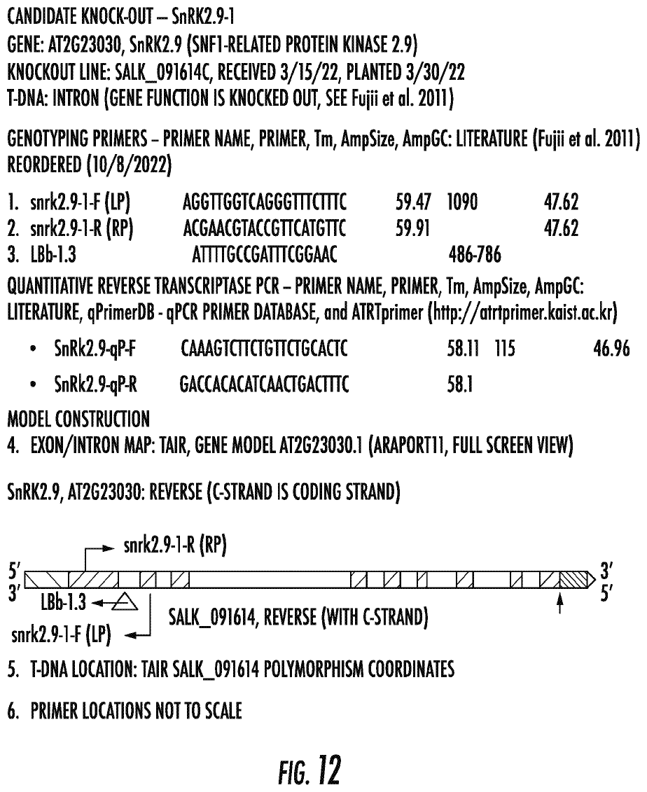

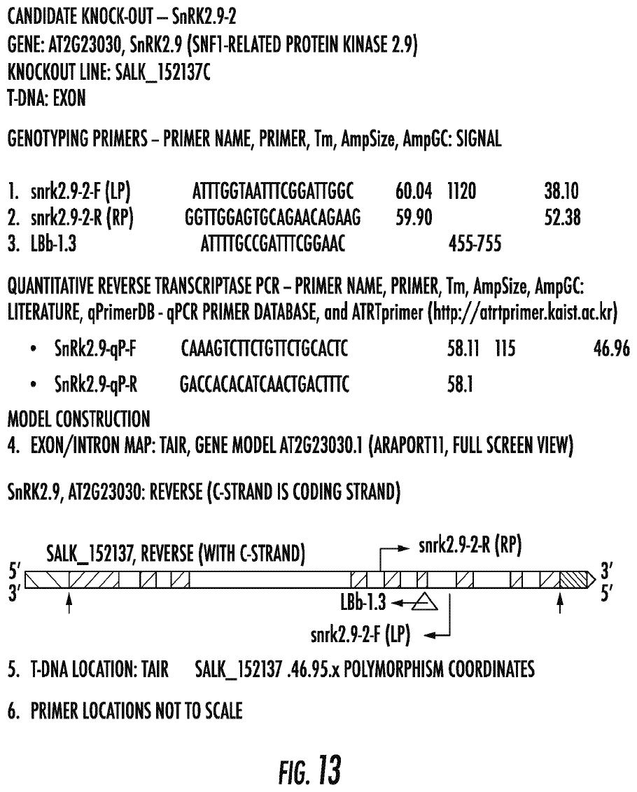

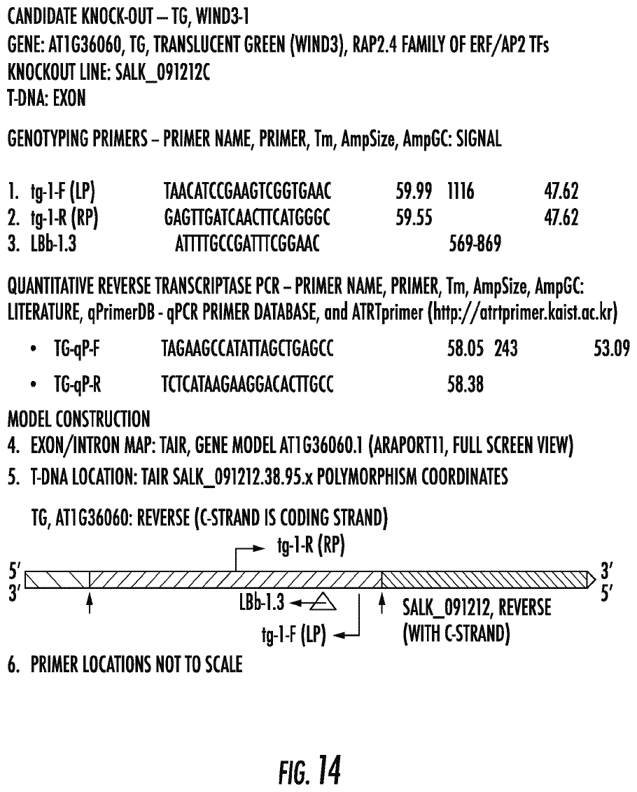

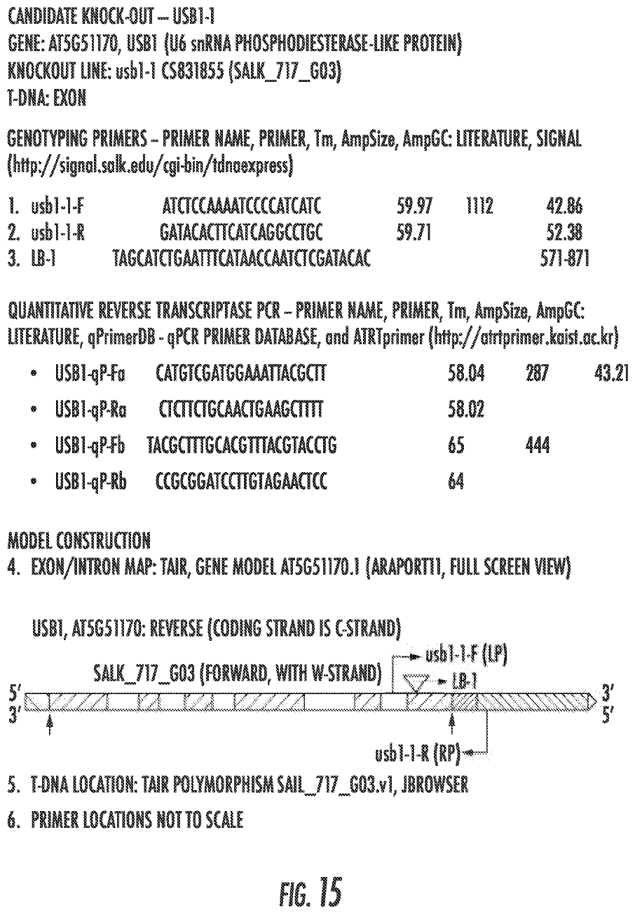

The present invention is the result of a series of experiments that tested the effects of down-regulating apomixis candidate genes on the initiation of unreduced ES formation. The candidates were identified from previously published [41] and unpublished expression profiling experiments. In each case, down-regulation was achieved by T-DNA inserted in the exon, intron or promoter regions of the candidate genes (FIG. 4-15).

### SUMMARY OF THE FINDINGS

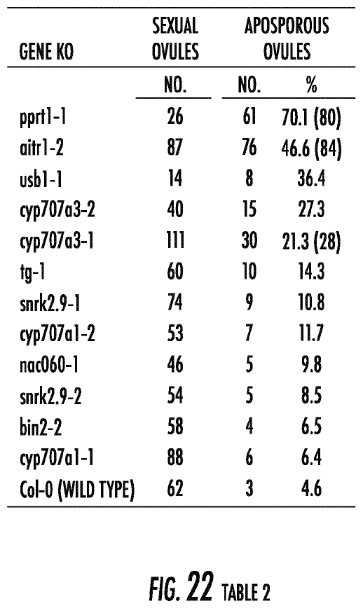

Each of the knocked out candidate gene lines described herein produced AIs or AESs in 6.4% to 84% of ovules (TABLE 2, FIG. 22).

### EXAMPLES

### Example 1. Switching from Megasporogenesis (Female Meiosis) and in ES Formation to 2n AES Formation in Sexual A. thaliana by Genetically Engineering the Down-Regulation Knock-Out of Certain Candidate Genes

### Materials and Methods

Knock-out lines of A. thaliana (FIG. 4-15), ecotype Columbia-0 (Col-0), were purchased from TAIR (https://www.arabidopsis.org/) and grown in controlled environment conditions as in [41]. To verify homozygous knock-out status, genomic leaf DNA was extracted (Genogrinder®; DNeasy Plant Pro Kit, www.qiagen.com). Primers for PCR are listed in TABLE 3. PCR reaction cycles (PCR Master Mix 2×, Thermo Scientific; 25 μL reactions) were: 98° C., 30 s; 94° C., 10 s, 54-55° C., 30 s; 72° C., 2 min; (94-72° C. cycled 31 times). PCR products were visualized in 1% agarose (1×TAE buffer, ethidium bromide, 120 v, 50 min).

For cytology, pistils were fixed, cleared, and observed by differential interference contrast microscopy as in [4]. The following characteristics were recorded: i) MMCs (without a large vacuole), ii) Antennaria type diplosporous 1-2 nucleate gametophytes (MMC with one or more large vacuoles), iii) sexual or Taraxacum type diplosporous dyads, iv) sexual tetrads, before or during degeneration of the three micropylar most spores and/or enlargement of the functional non-vacuolate megaspore, v) sexual 1-2 nucleate gametophytes (with one or more large vacuoles and degenerating tetrad remnants still visible); vi) Taraxacum type diplosporous 1-2 nucleate gametophytes (with one or more large vacuoles and the single degenerating spore still present), vii) AIs (enlarged non-vacuolate nucellar cells as large or larger than the sexual meiocyte or ES), viii) aposporous gametophytes (nucellar cells with one or more large vacuoles and one or more nuclei) adjacent to a degenerating tetrad with or without degeneration of the SES, and ix) status of the functional megaspore or young SES when one or more aposporous embryos are present. Pistil lengths corresponding to the following A. thaliana stages were recorded: MMC, meiosis, functional megaspore stage, and the early sexual gametophyte stage.

### Results

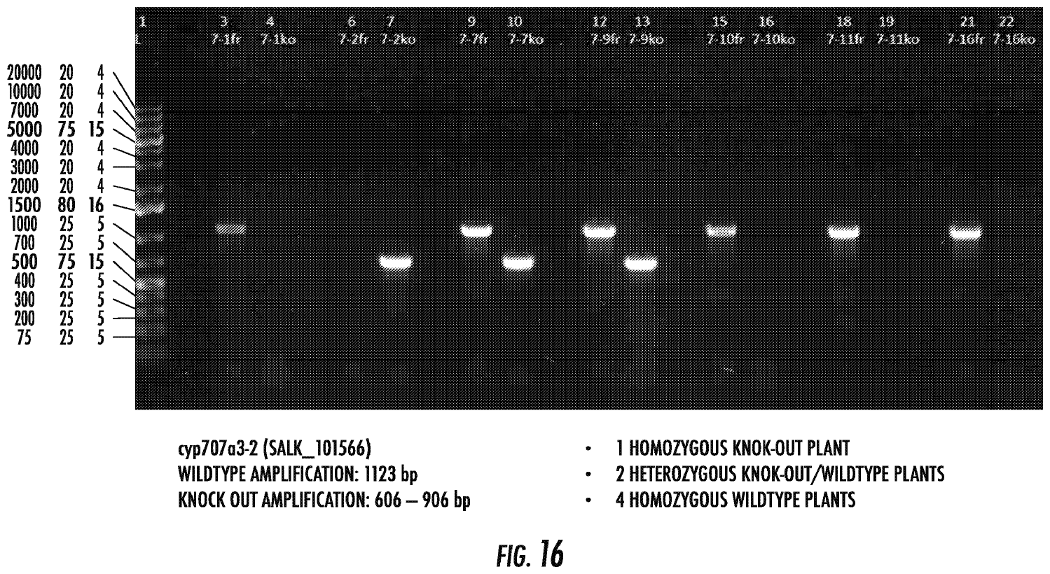

PCR verification of knock-out lines. All plants grown from TAIR knock-out lines (FIG. 4-15) were PCR verified. This was accomplished using a primer pair that amplified a specific length of each wild type gene only if the T-DNA was not present (wild type primer pair) and a second pair of primers that amplified part of the gene and part of the T-DNA insert (T-DNA knock-out primer pair). Accordingly, DNA gels with DNA from homozygous wild type or knock-out plants produced a single band (each type with a different position on the gel), while those with DNA from heterozygous plants produced both bands (from two separate PCR reactions). For example, DNA from seven putative cyp707a3 knock-out plants (FIG. 16) revealed one homozygous knock-out plant (lanes 6 and 7, used for cytological analyses), two heterozygous plants (lanes 9-13), and four wild type plants (lanes 3 and 4, lanes 15-22). We found that TAIR knock-out lines (FIG. 4-15) with a “C” following their genotype identifier number (TAIR indicator that the homozygous knock out line had been verified homozygous) were correct. Nevertheless, all plants were confirmed by PCR to be homozygous knock-outs, whether their TAIR number contained a C or not, before they were cytologically analyzed.

Aposporous ovules in wild type A. thaliana (Col-0) and in 12 knock-out lines of Col-0. To determine frequency of aposporous activity, ovules at the MMC stage through the functional megaspore stage of each knock-out line (FIG. 4-15) were examined cytologically by DIC microscopy. Ovules that contained either an AI or an AES were tabulated as aposporous ovules. The percentage of aposporous ovules was then tabulated for wild type Col-0 and 12 knock-out lines (TABLE 2, FIG. 22). Only three of 65 ovules from Col-0 wild type were aposporous (4.6%), and these were AIs. In comparison, over 20% of ovules in the knock-out lines of four genes (five lines) were aposporous, aitr1, pprt1, cyp707a3, and usb1, and many of the scored ovules contained cytologically distinct AESs. When the tabulation of aposporous ovules was restricted to ovule development stages where AIs and AESs generally form (tetrad stage through the early SES stage), the percentage of aposporous ovules increased to 84% in aitr1 (TABLE 2, FIG. 22).

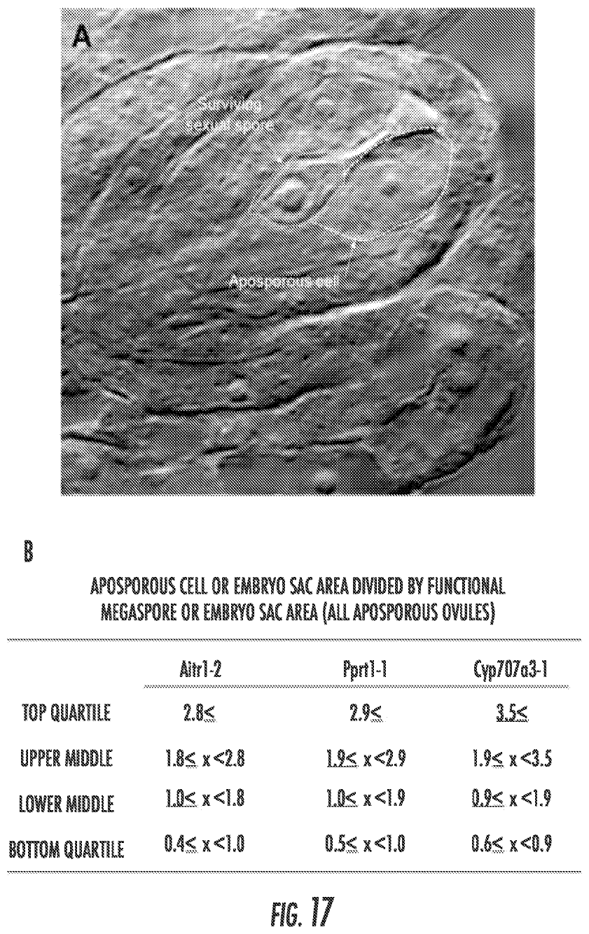

Competitiveness of aposporous development versus sexual development. To quantify the competitiveness of aposporous development (AIs or AESs), the 2-dimensional area of AIs or AESs in optical sagittal sections of ovules was divided by that of the sexual meiocyte (dyad or tetrad), enlarged functional megaspore, or early stage SES (FIG. 17 A, B). The resulting percentages were then partitioned into quartiles, with the largest 25% of values representing the top quartile, etc. Fifty percent of AIs or AESs in aitr1, pprt1, and cyp707a3 ovules ranged from 1.8 to 3.5 times larger than their corresponding sexual structures, and most of these larger volume structures were AESs (FIG. 17 B).

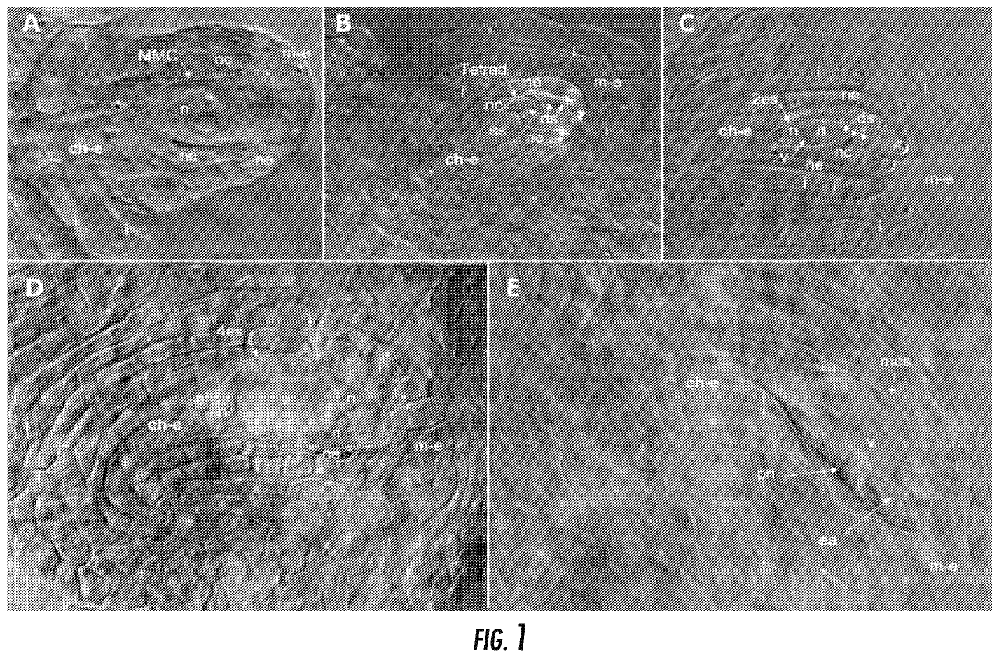

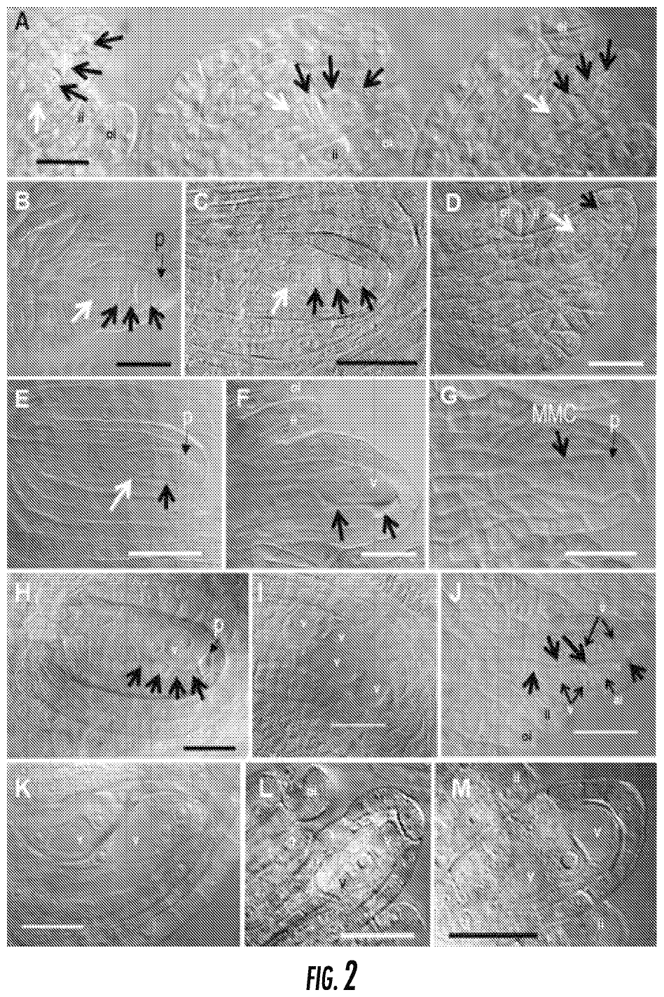

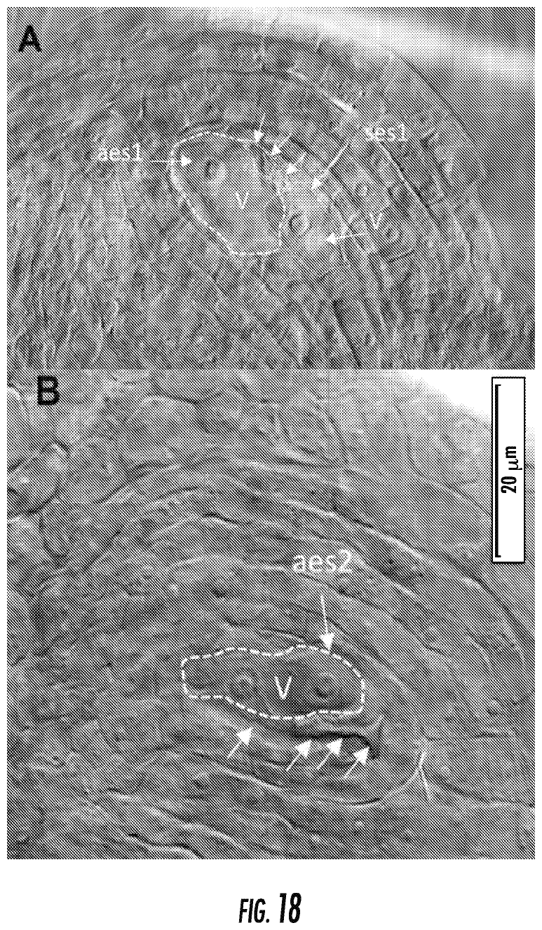

AESs replace SESs in ovules of cvp707a3 knock-out plants. In naturally occurring aposporously apomictic plants, large, healthy appearing and vacuolate 2n AESs frequently develop adjacent to either i) a large and healthy appearing 1n SES (with its three degenerating 1n megaspores) or ii) a tetrad in which all four 1n megaspores are degenerating. In the latter case, the sexual process completely fails, and there is no chance for a seed to form sexually. Such ovules will either produce clonal seeds by apospory, provided there are signals for parthenogenesis and endosperm formation, or they will produce empty seeds [4-6, 9](FIG. 1, 2). Development of AESs in cyp707a3 knock-out plants is identical to that observed in naturally aposporous plants, e.g., in the genus Boechera (compare AES formation in FIG. 2 to that shown in FIG. 18). Two developing 2n AESs in ovules of cyp707a3 knock-out plants are shown, a 1-nucleate AES (AES1) (FIG. 18 A) and a 2-nucleate AES (AES2) (FIG. 18 B). Next to the aes1 are three degenerating megaspores (unlabeled arrows) and a healthy looking and vacuolate 1-nucleate 1n SES. Note that the single nucleolus (prominent round dot) in the AES and SES often constitutes less than half of the nucleus area. The nucleus itself is bordered by a nuclear membrane that can be seen as a faint and continuous smooth circle surrounding the nucleolus and the nucleoplasm. Multiple nucleoli are often observed within the same nucleus, and individual nucleoli have sometimes been misinterpreted in published papers as multiple nuclei.

In some cases, a sexual and one or more AESs (commonly only multiple AESs) develop to maturity together in the same ovule (e.g., see FIG. 2 I, K). However, without a parthenogenesis component, only SESs will produce an embryo, endosperm and fertile seed. About 30% of cyp707a3 ovules were scored as aposporous (Table 2 (FIG. 22), producing AIs and/or AESs) and ca. ⅓ of these were AESs. Hence, while AESs form in these knock-out plants, aposporous seed set is not expected without a parthenogenesis signal. Even then, because the AES formation frequency is low (ca. 10%, lower if the SES displaces the AES), the percentage of seeds produced by apospory (coupled with parthenogenesis) will be low.

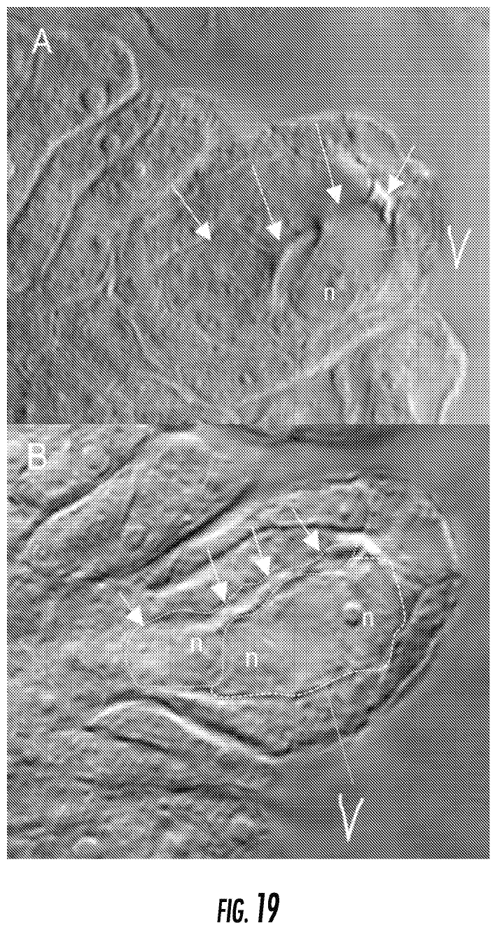

AESs replace SESs in ovules of pprt1 knock-out plants. 80% of pprt1 ovules were aposporous (Table 2, FIG. 22), and about ½ of these were AESs. These AESs developed as in cyp707a3, and as in other aposporous plants in general. Specifically, a large nucellar cell (AI) expanded further by forming a large vacuole, which is easily seen by DIC microscopy (FIG. 19 A). This AES1 along with other nucellar cells replaced the area previously occupied by the three tetrad megaspores programmed for degeneration. Remnant tissue from the degenerated megaspores is still visible at these early stages of AES and SES formation (arrows in FIG. 18, 19). In both ovules of FIG. 19 (A and B), the early SES, though still small and not as developed as the AESs, appears healthy and appears to be continuing to develop. Percentage values of pollination stage (mature) ovules containing AESs verses SESs is hard to determine because the origin, whether aposporous or sexual, of ESs is often difficult to determine beyond the 2-nucleate ES stage. This is because all tissue from degenerating megaspores at these more advanced stages of ovule and ES development has disintegrated. If there is only one ES present at these more advanced stages (the most common case), it is impossible to determine by DIC microscopy alone whether the ES is of sexual origin, having displaced the AES, or of aposporous origin [4-6, 9].

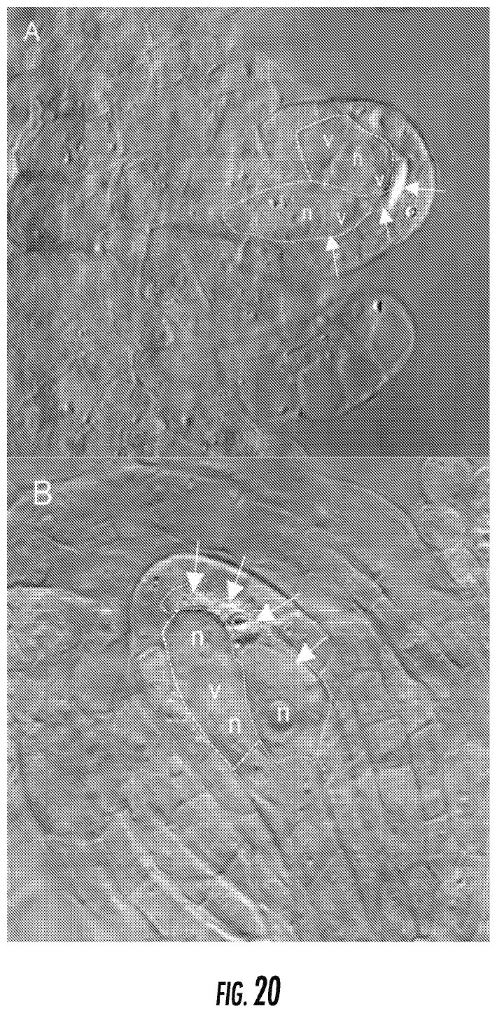

AESs replace SESs in ovules of aitr1 knock-out plants. 84% of aitr1 ovules were aposporous (Table 2, FIG. 22), and, as in ovules from pprt1, about ½ of these were AESs that developed following the typical pattern of AES formation in other aposporous plants. Again, example photomicrographs of an AES1 and an AES2 are displayed (FIG. 20 A, B).

AESs replace SESs in ovules of other knock-out lines. Though at lower frequencies (TABLE 2, FIG. 22), vacuolate and multinucleate AESs, typical of those shown in FIG. 18-20, replace SESs in ovules of usb1, tg-1, snrk2.9, cyp707a1, nac060 and bin2 knock-out plants.

### DISCUSSION

The present disclosure identifies nine specific genes that when downregulated produce AESs that replace SESs as shown herein through unambiguous photomicroscopic images. This is the first unambiguous disclosure of AESs being induced in sexual plants by genetic engineering. The genes chosen for study were identified based on the inventor's published expression profiling studies [41], more extensive yet to be published expression profiling studies, and published [41] and unpublished pharmacological studies. The latter were designed to test whether certain molecular pathways, identified through expression profiling studies of sexual and apomictic plants, affect sex apomixis switching in angiosperms. The pharmacological studies included important sexual crop and non-crop species from among the Brassicaceae (mustard, brassicas, etc.), Asteraceae (sunflowers, etc.), Poaceae (cereals and other grasses) and Fabaceae (legumes) families of angiosperms. In all cases, efficient aposporous and diplosporous 2n ES formation was induced by pharmacologically modifying the identified pathways ([41, 50]. From the many 1000s of genes that participate in these pathways, which could have been selected, the nine genes disclosed herein readily induce apospory in A. thaliana in a manner consistent with naturally occurring apomictic ES formation in species from 32 angiospermous orders, 78 angiospermous families and 293 angiospermous genera [2]. The molecular pathways altered by the nine genes disclosed herein occur in all angiosperms. That the pharmacological experiments have worked with all angiosperms attempted to date, is strong evidence that the genetic engineering approach for modifying these same pathways will also work in all angiosperms.

### REFERENCES

1. Hand M L, Koltunow A M: The genetic control of apomixis: asexual seed formation. Genetics 2014, 197(2):441-450.
2. Hojsgaard D, Klatt S, Baier R, Carman J G, Horandl E: Taxonomy and Biogeography of Apomixis in Angiosperms and Associated Biodiversity Characteristics. Critical Reviews in Plant Sciences 2014, 33(5):414-427.
3. Carman J G: Asynchronous expression of duplicate genes in angiosperms may cause apomixis, bispory, tetraspory, and polyembryony. Biological Journal of the Linnean Society 1997, 61(1):51-94.
4. Carman J G, Mateo de Arias M, Gao L, Zhao X, Kowallis B M, Sherwood D A, Srivastava M K, Dwivedi K K, Price B J, Watts L et al: Apospory and diplospory in diploid Boechera (Brassicaceae) may facilitate speciation by recombination-driven apomixis-to-sex reversals. Front Plant Sci 2019, 10:724.
5. Asker S E, Jerling L: Apomixis in Plants. Boca Raton, F L: CRC Press, Inc.; 1992.
6. Nogler G A: Gametophytic apomixis. In: Embryology of angiosperms. Edited by Johri B M. New York: Springer-Verlag; 1984: 475-518.
7. Crane C F: Classification of apomixis mechanisms. In: The Flowering of Apomixis: From Mechanisms to Genetic Engineering. Edited by Savidan Y, Carman J G, Dresselhaus T: Mexico, D.F.: CIMMYT, IRD, European Commission DGVI (FAIR); 2001: 24-43.
8. Johri B M, Ambegaokar K B, Srivastava P S: Comparative Embryology of Angiosperms Vol. 1. New York: Springer-Verlag; 1992.
9. Carman J G, Jamison M, Elliott E, Dwivedi K K, Naumova T N: Apospory appears to accelerate onset of meiosis and sexual embryo sac formation in sorghum ovules. BMC Plant Biol 2011, 11:9.
10. Schmidt A: Controlling Apomixis: Shared Features and Distinct Characteristics of Gene Regulation. Genes (Basel) 2020, 11(3).
11. Xu Y, Jia H, Tan C, Wu X, Deng X, Xu Q: Apomixis: genetic basis and controlling genes. Hortic Res 2022, 9:uhacl50.
12. Olmedo-Monfil V, Durin-Figueroa N, Arteaga-Vizquez M, Demesa-Arevalo E, Autran D, Grimanelli D, Slotkin R K, Martienssen R A, Vielle-Calzada J-P: Control of female gamete formation by a small RNA pathway in Arabidopsis. Nature 2010, 464(7288):628-632.
13. Schneitz K, Hulskamp M, Pruitt R E: Wild-type ovule development in Arabidopsis thaliana: a light microscope study of cleared whole-mount tissue. The Plant Journal 1995, 7(5):731-749.
14. Schmidt A, Wuest S E, Vijverberg K, Baroux C, Kleen D, Grossniklaus U: Transcriptome analysis of the Arabidopsis megaspore mother cell uncovers the importance of RNA helicases for plant germline development. PLoS Biol 2011, 9(9):e1001155.
15. Garcia-Aguilar M, Michaud C, Leblanc O, Grimanelli D: Inactivation of a DNA methylation pathway in maize reproductive organs results in apomixis-like phenotypes. Plant Cell 2010, 22(10):3249-3267.
16. Hernandez-Lagana E, Rodriguez-Leal D, Lua J, Vielle-Calzada J P: A Multigenic Network of ARGONAUTE4 Clade Members Controls Early Megaspore Formation in Arabidopsis. Genetics 2016, 204(3):1045-1056.
17. Colono C, Ortiz J P A, Permingeat H R, Souza Canada E D, Siena L A, Spoto N, Galdeano F, Espinoza F, Leblanc O, Pessino S C: A Plant-Specific TGS1 Homolog Influences Gametophyte Development in Sexual Tetraploid Paspalum notatum Ovules. Front Plant Sci 2019, 10:1566.
18. Mangelsdorf P C, Reeves R G: Hybridisation of maize, Tripsacum and Euchlaena. Journal of Heredity 1931, 22:329-343.
19. Rao N G P, Narayana L L: Apomixis in grain sorghums. Indian Journal of Genetics and Plant Breeding 1968, 28:121-127.
20. Asker S: Progress inn apomixis research. Hereditas 1979, 91:231-240.
21. Hanna W W, Bashaw E C: Apomixis—Its Identification and Use in Plant-Breeding. Crop Science 1987, 27(6):1136-1139.
22. Barcaccia G, Albertini E: Apomixis in plant reproduction: a novel perspective on an old dilemma. Plant Reprod 2013, 26(3):159-179.
23. Suomalainen E, Saura A, Lokki J: Cytology and Evolution in Parthenogenesis. Boca Raton, FL, USA: CRC Press, Inc.; 1987.
24. Bilinski C A, Marmiroli N, Miller J J: Apomixis in Saccharomyces cerevisiae and other eukaryotic micro-organisms. Adv Microb Physiol 1989, 30:23-52.
25. Grimanelli D: Epigenetic regulation of reproductive development and the emergence of apomixis in angiosperms. Curr Opin Plant Biol 2012, 15(1):57-62.
26. Neiman M, Sharbel T F, Schwander T: Genetic causes of transitions from sexual reproduction to asexuality in plants and animals. J Evol Biol 2014, 27(7):1346-1359.
27. Mogie M: The evolution of asexual reproduction in plants. London: Chapman & Hall; 1992.
28. Savidan Y: Apomixis: genetics and breeding. In: Plant Breeding Reviews. Edited by Janick J, vol. 18. New York: John Wiley & Sons, Inc.; 2000: 13-86.
29. Koltunow A M, Grossniklaus U: Apomixis: a developmental perspective. Annu Rev Plant Biol 2003, 54:547-574.
30. Ozias-Akins P, van Dijk P J: Mendelian genetics of apomixis in plants. Annu Rev Genet 2007, 41:509-537.
31. Vernet A, Meynard D, Lian Q, Mieulet D, Gibert O, Bissah M, Rivallan R, Autran D, Leblanc O, Meunier A C et al: High-frequency synthetic apomixis in hybrid rice. Nat Commun 2022, 13(1):7963.
32. Conner J A, Mookkan M, Huo H, Chae K, Ozias-Akins P: A parthenogenesis gene of apomict origin elicits embryo formation from unfertilized eggs in a sexual plant. Proc Natl Acad Sci USA 2015, 112(36):11205-11210.
33. Conner J A, Podio M, Ozias-Akins P: Haploid embryo production in rice and maize induced by PsASGR-BBML transgenes. Plant Reprod 2017, 30(1):41-52.
34. Carman J G: Apomixis for crop production: status of technology development and commercialization implications. Willamette Journal of International Law and Dispute Resolution 2004, 12:29-48.
35. d'Erfurth I, Jolivet S, Froger N, Catrice O, Novatchkova M, Mercier R: Turning meiosis into mitosis. PLoS Biol 2009, 7(6):e1000124.
36. Mieulet D, Jolivet S, Rivard M, Cromer L, Vernet A, Mayonove P, Pereira L, Droc G, Courtois B, Guiderdoni E et al: Turning rice meiosis into mitosis. Cell Res 2016, 26(11):1242-1254.
37. Khanday I, Skinner D, Yang B, Mercier R, Sundaresan V: A male-expressed rice embryogenic trigger redirected for asexual propagation through seeds. Nature 2019, 565(7737):91-95.
38. Underwood C J, Vijverberg K, Rigola D, Okamoto S, Oplaat C, Camp R, Radoeva T, Schauer S E, Fierens J, Jansen K et al: A PARTHENOGENESIS allele from apomictic dandelion can induce egg cell division without fertilization in lettuce. Nat Genet 2022, 54(1):84-93.
39. Taliaferro C M, Bashaw E C: Inheritance and control of obligate apomixis in breeding buffelgrass Pennisetum ciliare. Crop Science 1966, 6:473-480.
40. Van Dijk P J: Potential and realized costs of sex in dandelions, Taraxacum officinale s.l. In: Apomixis: evolution, mechanisms and perspectives. Edited by Hörandl E, Grossniklaus U, Van Dijk P J, Sharbel T F: ARG-Gantner, Ruggell; 2007.
41. Mateo de Arias M, Gao L, Sherwood D A, Dwivedi K K, Price B, Jamison M, Kowallis B, Carman J G: Whether gametophytes are reduced or unreduced in angiosperms might be determined metabolically. Genes 2020, 11(12):1449.
42. Albertini E, Barcaccia G, Carman J G, Pupilli F: Did apomixis evolve from sex or was it the other way around?Journal of Experimental Botany 2019, 70(11):2951-2964.
43. Mayr E: Animal Species and Evolution. Cambridge, M A: Belknap Press of Harvard University; 1963.
44. Crevillen P, Yang H, Cui X, Greeff C, Trick M, Qiu Q, Cao X, Dean C: Epigenetic reprogramming that prevents transgenerational inheritance of the vernalized state. Nature 2014, 515(7528):587-590.
45. Crisp P A, Ganguly D, Eichten S R, Borovitz J O, Pogson B J: Reconsidering plant memory: intersections between stress recovery, RNA turnover, and epigenetics. Science Advances 2016, 2(2):e1501733
46. Bonasio R: The expanding epigenetic landscape of non-model organisms. J Exp Biol 2015, 218(Pt 1):114-122.
47. Sommer R J: Phenotypic Plasticity: From Theory and Genetics to Current and Future Challenges. Genetics 2020, 215(1):1-13.
48. Boerman N A, Frei U K, Lubberstedt T: Impact of Spontaneous Haploid Genome Doubling in Maize Breeding. Plants (Basel) 2020, 9(3).
49. Sailer C, Schmid B, Grossniklaus U: Apomixis Allows the Transgenerational Fixation of Phenotypes in Hybrid Plants. Curr Biol 2016, 26(3):331-337.
50. Carman J G, Price B, Mateo de Arias M: Chemically induced apomictic embryo sac formation in sexual Arabidopsis, soybean and maize. In: American Society of Agronomy, Crop Science Society of America Annual Meetings: Nov. 9-12, 2021; Salt Lake City, U T. 2021.

What is claimed:

1. A method of inducing unreduced aposporous or diplosporous embryo sac formation in a sexual angiosperm comprising up regulation of the abscisic acid (ABA) signal transduction pathway.

2. The method of claim 1, wherein the up regulation comprises modifying the expression level of one or more genes selected from PPRT1, AITR1, USBI-1, CYP707a3 and CYP07a1 and orthologs thereof.

3. A method of inducing aposporous or diplosporous embryo sac formation in a sexual angiosperm comprising regulation of the sucrose non-fermenting-1-related 2 (SnRK2) signal transduction pathway.

4. The method of claim 3, wherein regulation of the SnRK2 signal involves modifying the expression of SnRK2.9 and orthologs thereof.

5. A method of inducing aposporous or diplosporous embryo sac formation in a sexual angiosperm comprising regulation of the expression of photosynthesis genes.

6. The method of claim 5, wherein regulation of photosynthesis gene expression involves modifying the expression of NAC60 and orthologs thereof.

7. A method of inducing aposporous or diplosporous embryo sac formation in a sexual angiosperm comprising regulation of the brassinosteroid signal transduction pathway.

8. The method of claim 7, wherein regulation of the brassinosteroid signal involves modifying the expression of BIN2 and orthologs thereof.

9. A method of inducing aposporous or diplosporous embryo sac formation in a sexual angiosperm comprising regulation of oxidative stress attenuation pathways.

10. The method of claim 9, wherein regulation of oxidative stress attenuation pathways involves modifying the expression of TG and orthologs thereof.

11. A method of inducing unreduced aposporous or diplosporous embryo sac formation in a sexual angiosperm comprising modifying the expression level of one or more genes selected from PPRT1, AITR1, USBI-1, CYP707a3 and CYP07a1 and orthologs thereof.

12. The method of claim 11, wherein modifying the expression level of the one or more genes reduces the expression of the gene.

13. The method of claim 11 or claim 12, wherein modifying the expression level of the one or more genes knocks out expression of the gene.

14. The method of any one of claims 11-13, wherein the one or more genes comprises PPRT1.

15. The method of any one of claims 11-14, wherein the one or more genes comprises AITR1.

16. The method of any one of claims 11-15, wherein the one or more genes comprises USBI-1.

17. The method of any one of claims 11-16, wherein the one or more genes comprises CYP07a3.

18. The method of any one of claims 11-17, wherein the one or more genes comprises CYP07a1.

19. The method of any one of claims 11-18, wherein the method is a step inducing apomixis.

20. The method of any one of claims 11-19, wherein the sexual angiosperm is selected from: alfalfa, amaranth, asparagus, barley, beans, beets, buckwheat, canary grass, cacao, carob, carrots, castor beans, chickpeas, chilis, clover, coffee, cotton, cowpea, cucumbers, cucurbits, durum, flax (linseed), fonio, Job's tears, kaniwa, lentils, lettuce, lupin beans, maize (corn), melons, mesquite, millet, oat, onions, peanuts, peas, peppers, pitseed goosefoot, quinoa, rapeseed, rice, rye, sorghum, soybean, spelt, squash, sunflower, tamarind, teff, tomato, triticale, turnips, wheat, and wild rice.

21. The method of any one of claims 11-19, wherein the sexual angiosperm plant is a member of the Brassicaceae, Fabaceae, Asteraceae, or Poaceae family.

22. The method of any one of claims 11-19, wherein method is used in haploid, diploid, polyploid or aneuploid plant reproduction.

23. The method of claim 22, wherein the plant is a triploid.

24. The method of claim 23, wherein the triploid is a banana plant.

25. The method of any one of claims 11-19, wherein the sexual angiosperm is part of a rice, corn, or wheat plant.

26. The method of any one of claims 11-19, wherein sexual angiosperm is part of a rose, petunia or lily plant.

27. The method of any one of claims 11-19, wherein the sexual angiosperm is part of a broccoli, kale, eggplant, tomato, pepper or sugarcane plant.

28. The method of any one of claims 11-27, wherein the method induces at least 5% unreduced aposporous or diplosporous embryo sac formation in the sexual angiosperm.

29. The method of any one of claims 11-27, wherein the method induces at least 20% unreduced aposporous or diplosporous embryo sac formation in the sexual angiosperm.

30. The method of any one of claims 11-27, wherein the method induces at least 50% unreduced aposporous or diplosporous embryo sac formation in the sexual angiosperm.

31. The method of any one of claims 11-27, wherein the method induces at least 75% unreduced aposporous or diplosporous embryo sac formation in the sexual angiosperm.

32. The method of any one of claims 11-27, wherein the method induces at least 90% unreduced aposporous or diplosporous embryo sac formation in the sexual angiosperm.

33. A method of inducing apomixis in flowering plants comprising:
(a) formation of an unreduced embryo sac that is genetically identical to the mother plant, the formation comprising inducing unreduced aposporous or diplosporous embryo sac formation by modifying the expression level of one or more genes selected from PPRT1, AITR1, USBI-1, CYP707a3, CYP07a1, SnRK2.9, NAC060, BIN2, TG and orthologs;
(b) development of a clonal embryo from the unreduced egg of the embryo sac by parthenogenesis; and
(c) formation of endosperm from the central cell of the embryo sac, which supplies nutrients to the embryo as it grows and develops into a seedling.

34. The method of claim 33, wherein modifying the expression level of the one or more genes reduces the expression of the gene.

35. The method of claim 33 or claim 34, wherein modifying the expression level of the one or more genes knocks out expression of the gene.

36. The method of any one of claims 33-35, wherein the flowering plant is selected from alfalfa, amaranth, asparagus, barley, beans, beets, buckwheat, canary grass, cacao, carob, carrots, castor beans, chickpeas, chilis, clover, coffee, cotton, cowpea, cucumbers, cucurbits, durum, flax (linseed), fonio, Job's tears, kaniwa, lentils, lettuce, lupin beans, maize (corn), melons, mesquite, millet, oat, onions, peanuts, peas, peppers, pitseed goosefoot, quinoa, rapeseed, rice, rye, sorghum, soybean, spelt, squash, sunflower, tamarind, teff, tomato, triticale, turnips, wheat, and wild rice.

37. A method of converting sexual crops to apomixis comprising inducing unreduced aposporous or diplosporous embryo sac formation by modifying the expression level of one or more genes selected from PPRT1, AITR1, USBI-1, CYP707a3 and CYP07a1 and orthologs.

38. The method of claim 37, wherein modifying the expression level of the one or more genes reduces the expression of the gene.

39. The method of claim 37 or claim 38, wherein modifying the expression level of the one or more genes knocks out expression of the gene.

40. The method of any one of claims 37-39, wherein the flowing plant is selected from alfalfa, amaranth, asparagus, barley, beans, beets, buckwheat, canary grass, cacao, carob, carrots, castor beans, chickpeas, chilis, clover, coffee, cotton, cowpea, cucumbers, cucurbits, durum, flax (linseed), fonio, Job's tears, kaniwa, lentils, lettuce, lupin beans, maize (corn), melons, mesquite, millet, oat, onions, peanuts, peas, peppers, pitseed goosefoot, quinoa, rapeseed, rice, rye, sorghum, soybean, spelt, squash, sunflower, tamarind, teff, tomato, triticale, turnips, wheat, and wild rice.
